(19) United States
(12) Patent Application Publication
(10) Pub. No.: US 2002/0127539 A1
(43) Pub. Date: Sep. 12, 2002

(54) Assays for identifying receptors having alterations in signaling

(76) Inventors: Alan S. Kopin, Wellesley, MA (US); Martin Beinborn, Boston, MA (US)

Correspondence Address: CLARK & ELBING LLP, 101 FEDERAL STREET, BOSTON, MA, 02110, US

(21) Appl. No.: 09/966,871

(22) Filed: Sep. 28, 2001

Related U.S. Application Data
(60) Provisional application No. 60/236,302, filed on Sep. 28, 2000.
(60) Provisional application No. 60/288,644, filed on May 3, 2001.

(51) Int. Cl. C12Q 1/00; C12Q 1/68; G01N 33/53; G01N 33/567

(52) U.S. Cl. 435/4; 435/6; 435/7.2

(57) ABSTRACT

The present invention provides methods of identifying receptors having altered signaling. In particular, the present invention provides an assay for the identification of receptors having alterations in ligand dependent or ligand independent signaling. Receptors having alterations in ligand dependent signaling that can be identified by the inventive method include hypersensitive hyposensitive receptors and receptors having increased or decreased potency. The inventive method is also applicable to the identification of receptors having alterations in basal activity, for example, constitutively active receptors or receptors having silencing mutations. Further applications include the identification of polymorphic or mutant receptors having alterations in signaling and receptors having an altered drug response. Finally, the receptors identified are useful as tools for drug discovery.

### CROSS REFERENCE TO RELATED APPLICATIONS

This application claims the benefit of the filing dates of provisional applications, U.S. Ser. No. 60/236,302, filed Sep. 28, 2000, and U.S. Ser. No. 60/288,644, filed May 3, 2001, hereby incorporated by reference.

### Statement as to Federally Sponsored Research

This application was supported in part by NIH grant DK46767. The government may have certain rights to this invention.

### BACKGROUND OF THE INVENTION

Receptors having altered signaling are important tools for drug discovery due to the fact that a considerable number of diseases and other adverse effects can result from abnormal receptor activity. The identification of receptors having altered signaling is also valuable in the identification of polymorphic receptors where the altered signaling contributes to disease. Similarly, it is important to identify mutant or polymorphic receptors where the mutation or polymorphism alters the response of the receptor to a particular ligand, for example, a drug or peptide hormone.

Receptors having altered signaling include receptors that display a change in ligand dependent or independent (basal) signaling. For example, ligand dependent receptors might display an increase or decrease in signaling. Ligand dependent receptors that have an increased sensitivity to ligand stimulation include hypersensitive receptors and receptors having increased potency. Alternatively, receptors having decreased sensitivity to ligand, or decreased potency, may be identified. In contrast, receptors that display an increase in basal activity are classified as constitutively active receptors. Receptors that have reduced basal activity are, for example, receptors having silencing mutations. Other receptors may in fact be non-functional, i.e., these have neither detectable basal or ligand induced activity.

Methods of identifying receptors having altered signaling that can be used in high throughput drug screening assays have been lacking. For example, it has been particularly challenging to identify receptors having alterations in basal signaling, for example, constitutively active receptors. Constitutively active receptors are particularly valuable as sensitive detection systems for drug discovery. There exists the need for a standardized screening assay for the routine identification of receptors having altered signaling, particularly receptors having an alteration in the level of basal signaling in the absence of ligand.

### SUMMARY OF THE INVENTION

The present invention provides a method of identifying a receptor (for example, a polymorphic receptor) having altered signaling by using an assay, preferably a transcriptional reporter assay, that can detect alterations in ligand dependent and ligand independent signaling of a receptor. The method involves comparing the signal generated by a candidate receptor to the signal generated by a negative control. A receptor having altered signaling is identified by detecting an increase or decrease in the level of ligand stimulated or basal activity of the candidate receptor, compared to the negative control, using the transcriptional reporter assay.

In a related embodiment, the present invention provides a method of identifying a receptor having altered signaling. The methods involves first identifying regions of homology between a wild-type receptor and at least one receptor having altered signaling. Mutations are then introduced into the wild-type receptor, the mutations being based on the region of homology between the wild-type receptor and the receptor having altered signaling, to yield a mutant receptor. An assay is then carried out to detect an alteration in signaling of the mutant receptor compared to the wild-type receptor. An increase or decrease in signaling in the mutant receptor, compared to the wild-type receptor, identifies the mutant receptor as a receptor having altered signaling.

The methods for detecting alterations in signaling, described above, are applicable in the detection of many kinds of altered signaling. For example, the methods are capable of detecting receptors having an increase or decrease in basal signaling, receptors having an increased or decreased sensitivity to ligand stimulation, receptors having increased or decreased potency, and even receptors that do not transmit a signal. The invention is particularly valuable because it has the ability to rapidly and reproducibly identify mutant and/or polymorphic receptors having such alterations in activity. Such mutant and polymorphic receptors having such alterations include G protein-coupled receptors (for example, G protein-coupled receptors coupled to Gαq, Gαs, or Gαi), transmembrane receptors, and nuclear receptors (for example, steroid hormone receptors). Once identified, such receptors can be further screened for an alteration in ligand induced response, for example, an altered response to a drug.

More particularly, the present invention provides a number of methods of identifying constitutively active receptors. In a first method, such receptors are detected by (1) identifying regions of homology between a nonconstitutively active receptor and at least one constitutively active receptor; (2) introducing mutations into the nonconstitutively active receptor, the mutations based on a region of homology between the nonconstitutively active receptor and the constitutively active receptor, to yield a mutant receptor; and (3) assaying the mutant receptor for increased basal activity compared to the nonconstitutively active receptor, an increase in basal activity in the mutant receptor compared to the nonconstitutively active receptor identifying the mutant receptor as a constitutively active receptor. Preferably the assay is a transcriptional reporter assay, for example, a luciferase assay or a chloramphenicol acetyl transferase assay.

In a related aspect, the present invention provides a second method of identifying a constitutively active receptor (for example, a polymorphic receptor) by (1) cotransfecting a first host cell with a reporter construct and an expression vector, the reporter construct including a response element and a promoter operably linked to a reporter gene, the response element being sensitive to a signal induced by the receptor, and the expression vector including a promoter operably linked to the candidate receptor; (2) cotransfecting a second host cell with the reporter construct and a negative control vector; and (3) measuring the basal level of expression of the reporter construct in the first host cell and the second host cell, an increased basal level of expression in the first host cell compared to the second host cell identifying the candidate receptor as a constitutively active receptor.

The methods of identifying constitutively active receptors described herein are useful for identifying constitutively active G protein-coupled receptors, particularly G protein-coupled receptors that are coupled to Gαq, Gαs, or Gαi. Alternatively, the methods relate to the identification of a constitutively active single transmembrane receptor, for example, a constitutively active erythropoietin receptor. In another preferred embodiment, the methods relate to the identification of a constitutively active nuclear receptor, for example, a constitutively active steroid hormone receptor.

The particular response element used in the assay of the invention may be any response element that is sensitive to signaling through a particular receptor. Examples of preferred response elements include a portion of the somatostatin promoter (which has included a number of different response elements) (SMS), the serum response element (SRE), and the cAMP response element (CRE), which are response elements sensitive to G protein-coupled receptor signaling. Other preferred response elements include response elements sensitive to signaling through a single transmembrane receptor or a nuclear receptor.

In another aspect, the invention provides a general method of identifying a G protein-coupled receptor with altered signaling, by co-transfecting a first host cell with a reporter construct, the reporter construct including a G protein response element and a promoter operably linked to a reporter gene, a first expression vector, the first expression vector including a promoter operably linked to a candidate G protein-coupled receptor, and a second expression vector, the second expression vector including a promoter operably linked to a chimeric G protein, where the chimeric G protein is capable of receiving a signal from the candidate G protein-coupled receptor and increasing the expression of the reporter construct; co-transfecting a second host cell with the reporter construct, the second expression vector, and a negative control vector; and measuring the level of expression of the reporter construct in the first host cell and the second host cell, where an increased or decreased level of expression in the first host cell compared to the second host cell identifies the candidate receptor as a G protein-coupled receptor with altered signaling.

In an embodiment of this second aspect, the chimeric G protein includes a G protein with the C-terminal 3 amino acids changed to those of another G protein. In another embodiment of this second aspect, the chimeric G protein can be Gq5i, Gq5o, Gq5z, Gq5s, Gs5q, or G13Z. The reporter construct can be a luciferase construct, a beta-galactosidase construct, or a chloramphenicol acetyl transferase construct. The response element can be the somatostatin promoter, the serum response element, or the cAMP response element.

In other embodiments of the invention, the G protein coupled receptor can be a constitutively active receptor, a hypersensitive receptor, a hyposensitive receptor, a non-functional receptor, a silent receptor, or a partially silent receptor. In other embodiments of the invention, the G protein-coupled receptor can be coupled to a G protein, for example, Gαq, Gαs, Gαi, or Go. The signaling can be ligand dependent signaling or ligand independent signaling. In another embodiment of this aspect, the receptor with altered signaling can be further screened for an alteration in a response induced by a ligand. The ligand can be a drug, an agonist, an antagonist, or an inverse agonist.

In addition, it will be appreciated that the signaling detected by the particular response element can be any receptor signaling, including increased basal signaling (constitutive signaling), decreased basal signaling (silencing), and hypersensitive as well as hyposensitive signaling.

In a final preferred embodiment, the present invention provides a database that includes a collection of sequences of receptor polypeptides that exhibit alterations in signaling. This database need not be a static data base, but cab be a database that is forever increasing in size as additional polypeptides exhibiting alterations in signaling are identified and added to the collection. Preferably the database has 100 to 1000 sequences. In this way the database is continually improved over time.

Receptor polypeptides that make up the database may include receptors having alterations in ligand dependent or ligand independent signaling. Such receptor polypeptides may include G protein-coupled receptors, single transmembrane receptors, and nuclear receptors. Within a defined collection of G protein-coupled receptors, single transmembrane receptors, or nuclear receptors, the collection may be further defined as a collection of G protein-coupled receptors, single transmembrane receptors, or nuclear receptors that are constitutively active, silenced, hypersensitive, non-functional, or have an increased or decreased potency. Such receptors may, of course, be wild-type, mutant, or polymorphic polypeptide receptors.

By a “constitutively active receptor” is meant a receptor with a higher basal activity level than the corresponding wild-type receptor or a receptor possessing the ability to spontaneously signal in the absence of activation by a positive agonist. This term includes wild-type receptors that are naturally constitutively active (e.g., naturally occurring receptors, including naturally occurring polymorphic receptors and wild-type receptors) and that have a higher basal activity level than a corresponding vector lacking a gene encoding a receptor. The constitutive activity of a receptor may also be established by comparing the basal level of signaling, such as second messenger signaling, of a mutant receptor to the basal level of signaling of the wild-type receptor. A constitutively active receptor exhibits at least a 25% increase in basal activity, preferably, at least a 50% increase in basal activity, more preferably at least a 75% increase in basal level activity, and, most preferably more than a 100% increase in basal level activity, compared to either the negative control or the wild-type receptor. It is common for a constitutively active receptor, e.g., a polymorphic constitutively active receptor, that is associated with a disease phenotype, to display a relatively small increase in constitutive activity (e.g., as little as a 25% increase). Preferably, the basal activity of a constitutively active receptor can be confirmed by its decrease in the presence of an inverse agonist.

“Basal” activity means the level of activity (e.g., activation of a specific biochemical pathway or second messenger signaling event) of a receptor in the absence of stimulation with a receptor-specific ligand (e.g., a positive agonist). Preferably, the basal activity is less than the level of ligand-stimulated activity of a wild-type receptor. However, in certain cases, a mutant receptor with increased basal activity might display a level of signaling that approximates, is equal to, or even exceeds the level of ligand-stimulated activity of the corresponding wild-type receptor.

A “naturally-occurring” receptor refers to a form or sequence of a receptor as it exists in an animal, or to a form of the receptor that is homologous to the sequence known to those skilled in the art as the “wild-type” sequence. Those skilled in the art will understand “wild type” receptor to refer to the conventionally accepted “wild-type” amino acid consensus sequence of the receptor, or to a “naturally-occurring” receptor with normal physiological patterns of ligand binding and signaling.

A “mutant receptor” is understood to be a form of the receptor in which one or more amino acid residues in the predominant receptor occurring in nature, e.g., a naturally-occurring wild-type receptor, have been either deleted or replaced. Alternatively additional amino acid residues have been inserted.

By “altered signaling” is meant a change in the ligand dependent or ligand independent signal typically generated by a receptor, as measured by the parameters of efficacy, potency, or basal signaling. The change or alteration may be an increase or decrease in ligand dependent or ligand independent signaling. Examples of alterations in signaling include receptors having an increased sensitivity to ligand, i.e., hypersensitive receptors. This increased sensitivity to ligand may occur in the form of increased potency or increased efficacy in response to agonist stimulation. Other examples of receptors having alterations in signaling include receptors exhibiting a decreased sensitivity to ligand (i.e., hyposensitive or silenced receptors), receptors exhibiting a change in basal activity (e.g., receptors having an increased level of basal signaling, such as constitutively active receptors, or receptors having a decreased level of basal signaling, such as receptors having silencing mutations, i.e., fully silenced or partially silenced receptors). The change or alteration in signaling may also be an absence of signaling, for example, a non-functional receptor that does not bind a ligand, or a receptor that binds a ligand but does not transduce a ligand induced signal. A receptor with altered signaling exhibits at least a 25% increase or decrease in basal activity, or at least a 50% increase or decrease in basal activity, or at least a 75% increase or decrease in basal activity, or more than a 100% increase or decrease in basal activity, compared to an appropriate negative control. Alternatively, or in addition, a receptor with altered basal signaling exhibits at least a 5% increase or decrease, or at least a 10%, 15%, 20%, or 25% increase or decrease, or at least a 50%, 60%, or 75% increase or decrease, or more than a 100% increase or decrease in basal activity when expressed as a percentage of the hormone-induced maximal activity, all compared to an appropriate negative control. At the very least, a receptor with altered signaling exhibits a change in basal or ligand induced signaling or efficacy or potency relative to an appropriate negative control that is considered statistically significant using accepted methods of statistical analysis.

By “substantially pure nucleic acid” is meant nucleic acid (e.g., DNA or RNA) that is free of the genes which, in the naturally-occurring genome of the organism from which the DNA of the invention is derived, flank the gene. The term therefore includes, for example, a recombinant DNA which is incorporated into a vector; into an autonomously replicating plasmid or virus; or into the genomic DNA of a prokaryote or eukaryote; or which exists as a separate molecule (e.g., a cDNA or a genomic or cDNA fragment produced by PCR or restriction endonuclease digestion) independent of other sequences. It also includes a recombinant DNA which is part of a hybrid gene encoding additional polypeptide sequence.

“Transformed cell” means a cell into which (or into an ancestor of which) has been introduced, by means of recombinant DNA techniques, a DNA molecule encoding (as used herein) a polypeptide described herein (for example, a mu opioid receptor polypeptide).

“Promoter” means a minimal sequence sufficient to direct transcription. Also included in the invention are those promoter elements which are sufficient to render promoter-dependent gene expression controllable for cell-type specific or tissue-specific regulators; or inducible by external signals or agents; such elements may be located in the 5′ or 3′ regions of the native gene. A promoter element may be positioned for expression if it is positioned adjacent to a DNA sequence so it can direct transcription of the sequence.

“Operably linked” means that a gene and a regulatory sequence(s) are connected in such a way as to permit gene expression when the appropriate molecules (e.g., transcriptional activator proteins) are bound to the regulatory sequence(s).

“Expression vectors” contain at least a promoter operably linked to the gene to be expressed.

A “reporter construct” includes at least a promoter operably linked to a reporter gene. Such reporter genes may be detected directly (e.g., by visual inspection or detection through an instrument) or indirectly (e.g., by binding of an antibody to the reporter gene product or by reporter product-mediated induction of a second gene product). Examples of standard reporter genes include genes encoding the luciferase, green fluorescent protein, or chloramphenicol acetyl transferase gene polypeptides (see, for example, Sambrook, J. et al., Molecular Cloning: a Laboratory Manual, Cold Spring Harbor Press, N.Y., or Ausubel et al., Current Protocols in Molecular Biology, Greene Publishing Associates, New York, N.Y., V 1-3, 2000, incorporated herein by reference). Expression of the reporter gene is detectable by use of an assay that directly or indirectly measures the activity of the polypeptide encoded by the reporter gene. Preferred reporter constructs also include a response element.

A “response element” is a nucleic acid sequence that is sensitive to a particular signaling pathway, e.g., a second messenger signaling pathway, and assists in driving transcription of the reporter gene. According to the present invention, the response element may be the promoter.

As used herein, “second messenger signaling activity” refers to production of an intracellular stimulus (including, but not limited to, cAMP, cGMP, ppGpp, inositol phosphate, or calcium ions) in response to activation of the receptor, or to activation of a protein in response to receptor activation, including but not limited to a kinase, a phosphatase, or to activation or inhibition of a membrane channel.

A “negative control,” as used herein, is any construct that can be used to distinguish alterations in the signaling of a candidate receptor. The appropriate negative control for any given candidate receptor will vary depending on the assay and the type of alteration in signaling. For example, to identify a constitutively active receptor, the appropriate negative controls may be a vector lacking any receptor nucleotide sequences or a vector including non-constitutively active wild type receptor nucleotide sequences. The appropriate negative control to be used to identify a receptor with altered signaling will be apparent to a person of ordinary skill in the art.

### BRIEF DESCRIPTION OF THE DRAWINGS

FIG. 1 is a table of constitutively active Class I G protein-coupled receptors (SEQ ID NOS: 2-75). The mutations that impart constitutive activity to the receptors are indicated.

FIG. 2 is a graph showing the constitutive activity of the L325E CCK-BR receptor as assayed using a luciferase reporter assay.

FIG. 3 is a graph showing the constitutive activity of the Asn150Ala rat mu opioid receptor as assayed using a luciferase reporter assay. This is evidenced by the following: (1) agonist (DAMGO) stimulation of the receptor leads to a decrease in forskolin induced activity, indicating that the receptor works through an inhibiting pathway; (2) forskolin induced activity in the absence of DAMGO is lower with coexpression of mutant receptor (vs. wild-type receptor), indicating ligand independent activity of the inhibitory pathway.

FIG. 4 is a graph showing the effects of forskolin stimulation on HEK293 cells transfected with pcDNA1 and a CRE-Luc reporter construct.

FIG. 5 is a graph showing the sensitivity of the reporter constructs, SMS-Luc, SRE-Luc, and SRE-Luc+Gq5i to ligand-mediated activation of the mu opioid receptor.

FIG. 6 is a graph showing the constitutive activity of the Asn150Ala rat mu opioid receptor as assayed using the SRE-Luc/Gq5i luciferase reporter assay.

FIG. 7 is an illustration of a seven transmembrane domain Class I G protein-coupled receptor. Selected residues are indicated.

FIG. 8 is an illustration showing the amino acid residues conserved between the mu opioid receptor, the bradykinin B2 receptor, and the angiotensin II AT1A receptor.

FIG. 9 is an illustration showing the amino acid residues conserved between the oxytocin, vasopressin-V2, cholecystokinin-A, melanocortin-4, and α1b adrenergic receptors.

FIG. 10 is a graph showing the constitutive activity of the D146M MC-4 receptor as assayed using a luciferase reporter assay.

FIG. 11 is an illustration showing the positions relative to the CWLP motif (positions -13 and -20) conserved between the 1A adrenergic receptor, the α2C adrenergic receptor, the β2 adrenergic receptor, the serotonin 2A receptor, the cholecystokinin-B receptor, the platelet activating factor receptor, and the thyroid stimulating hormone receptor. (Conserved residues are indicated by a single letter code.)

FIG. 12 is an illustration showing a sequence alignment of the human kappa opioid receptor (ork), the rat kappa opioid receptor (orkr), the human mu opioid receptor (orm), the rat mu opioid receptor (ormr), the human delta opioid receptor (ord), the rat type 1A angiotensin II receptor (AT1A), and the human bradykinin receptor (B2) (SEQ ID NOS: 76-82).

FIG. 13 is an illustration showing the amino acid sequence (top to bottom) of the mouse mu opioid receptor, the rat mu opioid receptor, the bovine mu opioid receptor, the human mu opioid receptor, the pig mu opioid receptor, the white sucker (ws) opioid receptor, the angiotensin AT-1 receptor, and the bradykinin-B2 receptor (SEQ ID NOS: 83, 79, 84-87, 81, and 82).

### DESCRIPTION OF THE PREFERRED EMBODIMENTS

The present invention provides a rapid and reproducible screening assay for the detection of alterations in the signaling activity of a receptor. The assay may be applied to receptors with known ligands, as well as to receptors for which the ligand is presently unknown (i.e., orphan receptors). The assay may also be applied to polymorphic receptors. In one preferred embodiment, the screening assay is used to detect alterations in the basal level of signaling of a receptor. According to the present invention, receptors with increased basal level signaling are identified as constitutively active receptors. Constitutively active receptors include constitutively active G protein-coupled receptors (e.g., opiate receptors), single transmembrane domain receptors (e.g., the erythropoietin receptor (EPO receptor)), and nuclear receptors (e.g., steroid hormone receptors, such as the estrogen receptor). In another preferred embodiment, the screening assay is used to detect a decrease in the basal level signaling of a particular (e.g., naturally occurring constitutively active) receptor, for example, receptors having silencing mutations. In yet another preferred embodiment, the alteration in signaling is an alteration that results in a hypersensitivity to ligand stimulation.

According to the present invention, constitutively active receptors include naturally occurring constitutively active receptors and non-naturally occurring (i.e., mutant) constitutively active receptors. The present invention provides methods of identifying both naturally and non-naturally occurring constitutively active receptors. According to the present invention, constitutively active receptors with increased basal activity are compared to the appropriate negative control. For example, naturally occurring constitutively active receptors can be identified by exhibiting an increased basal level of signaling compared to the activity of a vector lacking a gene encoding a receptor. Alternatively, mutant receptors having constitutive activity can be identified by comparing the basal level of signaling of the mutant constitutively active receptor to the basal level of signaling of the wild-type receptor. An increase (e.g., by at least 25%) in basal level activity in a candidate receptor compared to a control or wild-type receptor indicates identification of a constitutively active receptor.

Many naturally occurring and non-naturally occurring constitutively active receptors have been previously identified and are available in the art. As described herein, this information can be harnessed and used as a tool to identify additional constitutively active receptors. According to the present invention, the amino acid and/or nucleic acid sequences of known constitutively active receptors are assembled into a database. The assembled database is then used to identify conserved domains that are important for constitutive activity, or to identify mutations within those domains that impart constitutive activity onto a particular receptor. The sequences of constitutively active polypeptides in such a database (including both naturally occurring constitutively active receptors and mutant receptors having constitutive activity) are then compared to the sequence of a given non-constitutively active receptor and conserved domains are identified between the nonconstitutively active receptor and the constitutively active receptors. This information is further used to identify specific residues within a given nonconstitutively active (e.g., wild-type) receptor that are likely to impart constitutive activity to the nonconstitutively active receptor upon mutation.

Once specific positions in a given nonconstitutively active receptor are targeted for mutation, receptors containing the identified mutations are generated using routine methods and screened for increased constitutive activity (see, for example, Sambrook, J. et al., Molecular Cloning: a Laboratory Manual, Cold Spring Harbor Press, N.Y., or Ausubel et al., Current Protocols in Molecular Biology, Greene Publishing Associates, New York, N.Y., V 1-3, 2000, incorporated herein by reference). Preferably, an increase in basal level activity is detected by measuring an increase in basal level signaling in the mutant receptor, compared to the wild-type receptor. The skilled artisan will appreciate that any assay typically used for measuring the ligand-stimulated activity of the wild-type receptor may also be used to measure the basal level activity of a mutant receptor. Such assays are discussed in further detail herein, below.

Those skilled in the art will appreciate that the basic principles that apply to the identification of receptors having increased basal level activity (constitutively active receptors) are directly applicable to the identification of receptors having reduced basal level activity (e.g., receptors having silencing mutations) and also to receptors that are hypersensitive.

One skilled in the art would clearly understand that in order to identify receptors having silencing mutations, one would screen for receptors having a decreased level of basal activity, rather than an increased level of basal activity. Hypersensitive receptors are similarly identified. Hypersensitive receptors are receptors that deliver an increased receptor induced signal in response to a ligand, compared to the wild-type receptor. In preferred embodiments, non-naturally occurring receptors that are hypersensitive are identified by comparing the ligand-induced activity of the wild-type receptor to the ligand-induced activity of the mutant receptor; a hypersensitive receptor being identified by its ability to display a stronger signal to a given concentration of ligand than the wild-type receptor. A hypersensitive receptor may be characterized in that it exhibits an increased response to a specific concentration of ligand, compared to the response of a wild-type receptor to the same concentration of ligand. For example, if 5 μM ligand induces a 5-fold stimulation of activity in a wild-type receptor, compared to a negative control, 5 μM ligand may stimulate a 10-fold stimulation in activity in a hypersensitive receptor, compared to the same negative control.

### Identifying Receptors Having Altered Signaling

The present invention provides a method of identifying constitutively active receptors. As noted above, some receptors (e.g., wild-type receptors) are naturally constitutively active. Such naturally occurring constitutively active receptors are identified by simply comparing the basal activity of the wild-type receptor to that of a negative control. A suitable negative control is, for example, a cell lacking expression of the natural wild-type receptor (e.g., a cell transfected with an empty expression vector, a cell transfected with a wild-type vector, or a cell transfected with a different receptor that has been previously established to lack constitutive activity (preferably both an empty expression vector and a wild-type vector are used)). Alternatively, the present invention provides a method of identifying mutation-induced constitutively active receptors. Preferably, the mutation-induced constitutively active receptors are receptors of therapeutic interest. According to the present invention, mutation-induced constitutively active receptors may be identified systematically by (1) identifying regions of homology between a nonconstitutively active wild-type receptor and one or more constitutively active receptors; (2) introducing mutations into one or more regions of the nonconstitutively active receptor based on the identified region(s) of homology; and (3) assaying the mutant receptors for constitutive activity. Methods of achieving each of these steps are described in detail below.

One skilled in the art will appreciate that the mutations can be introduced by any random mutagenesis procedure standard in the art. A large variety of random mutagenesis kits are in fact commercially available. Once identified, the constitutive activity of the receptor may be confirmed, for example, using a mammalian expression system, particularly a yeast expression system.

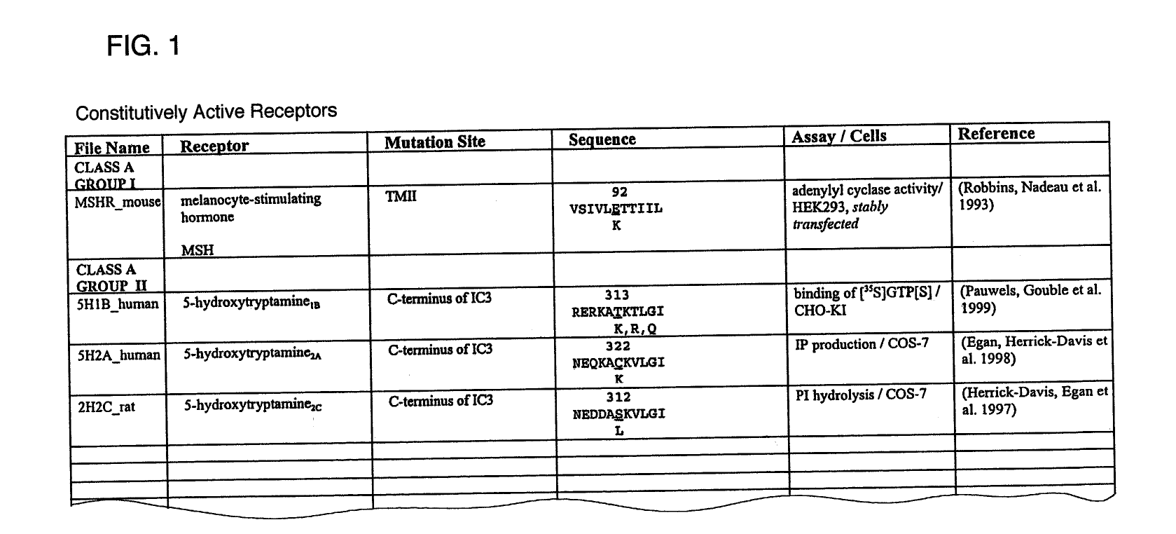

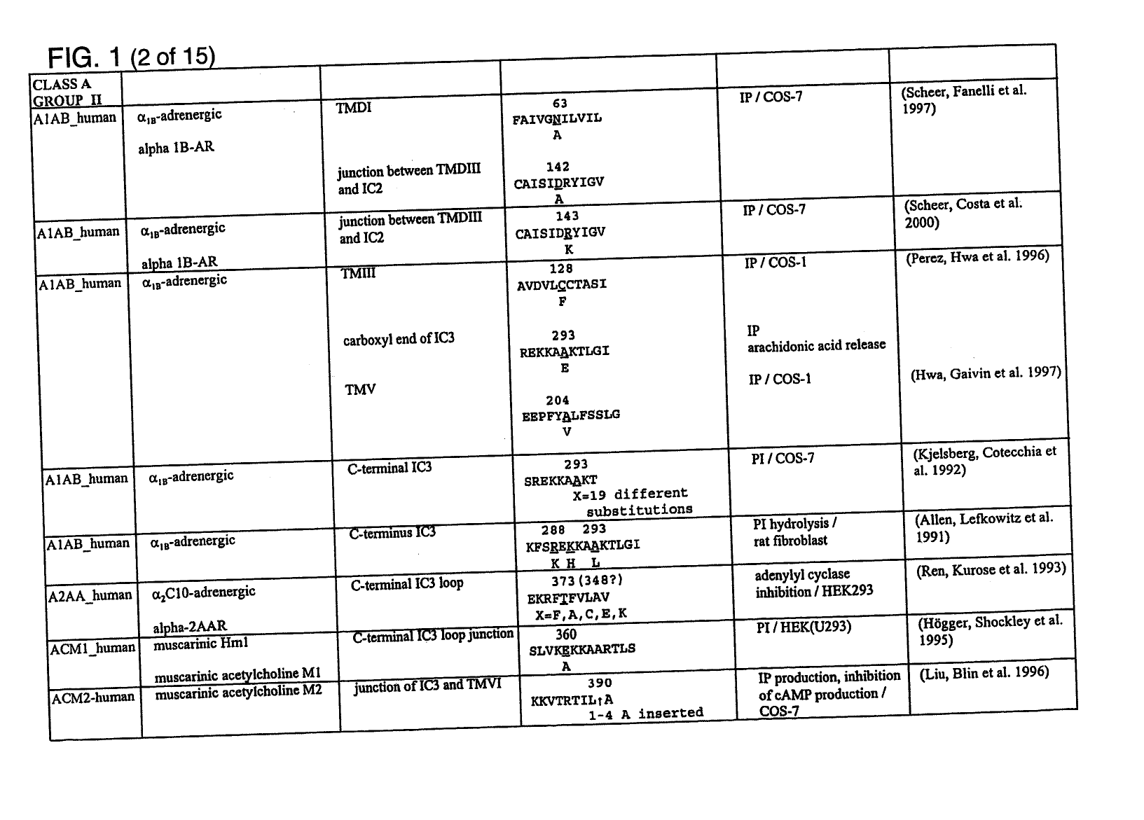

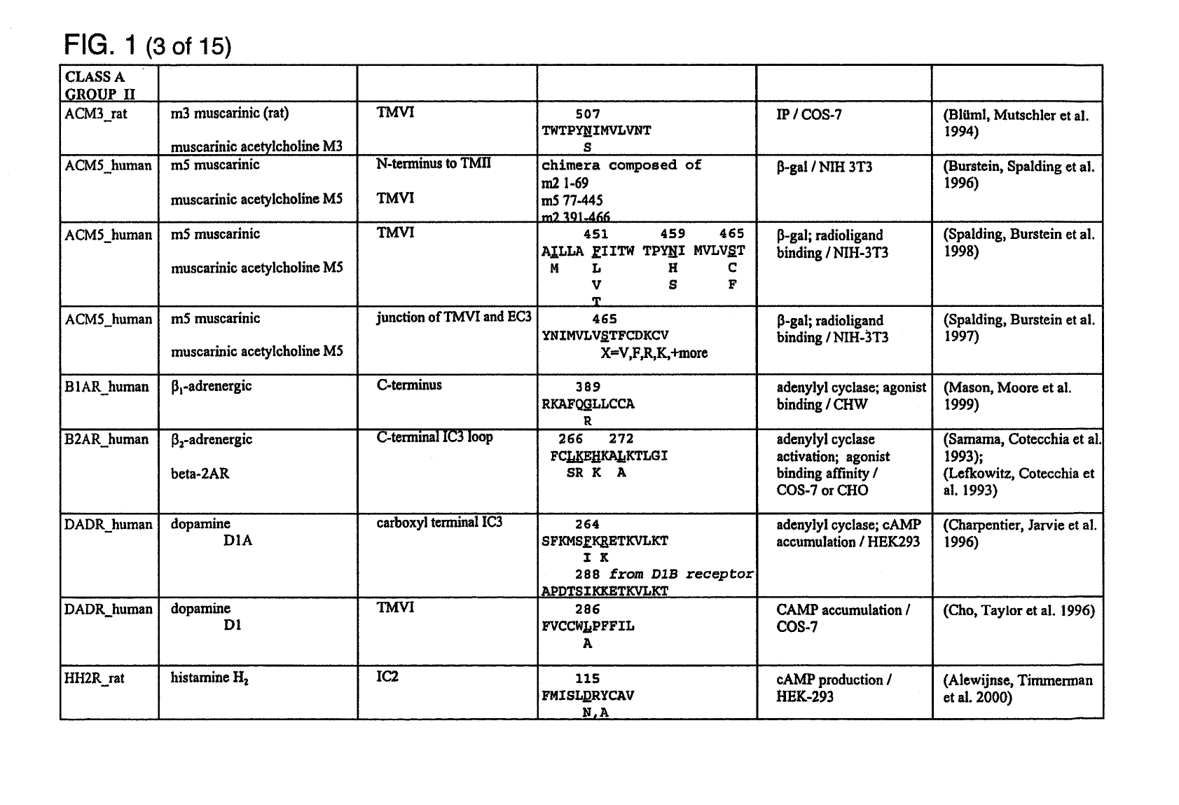

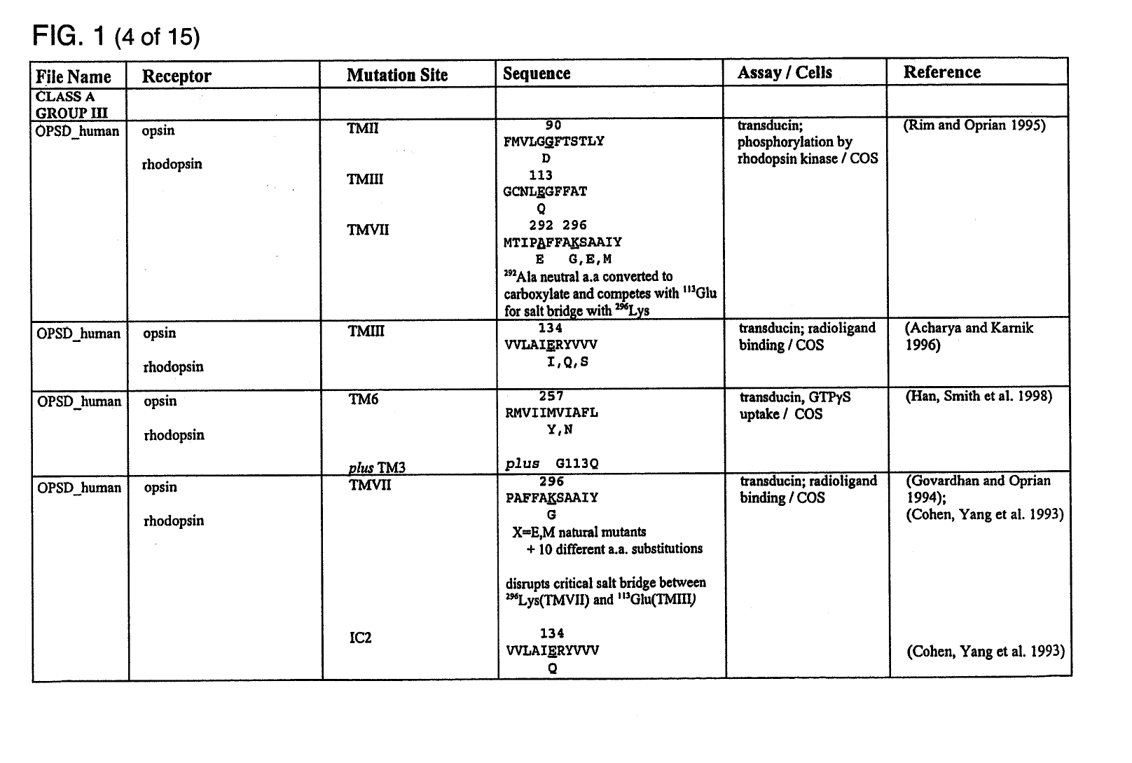

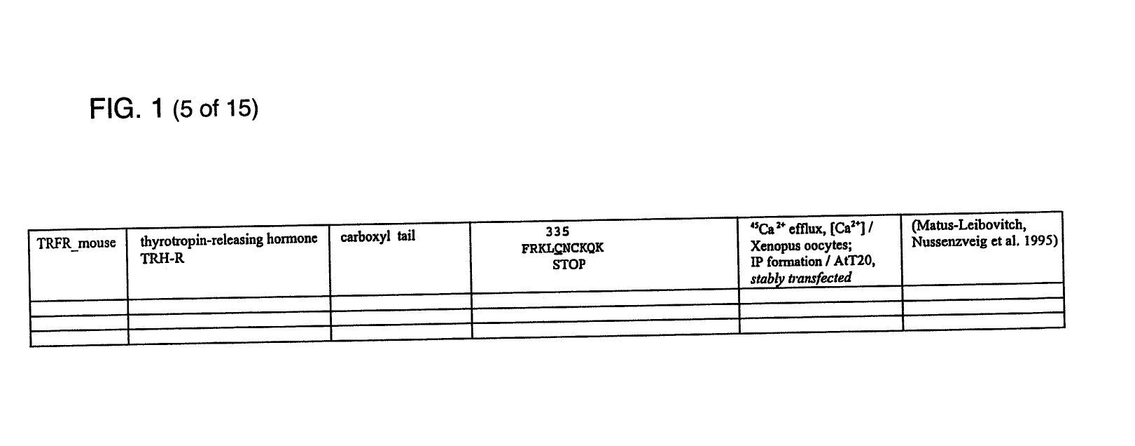

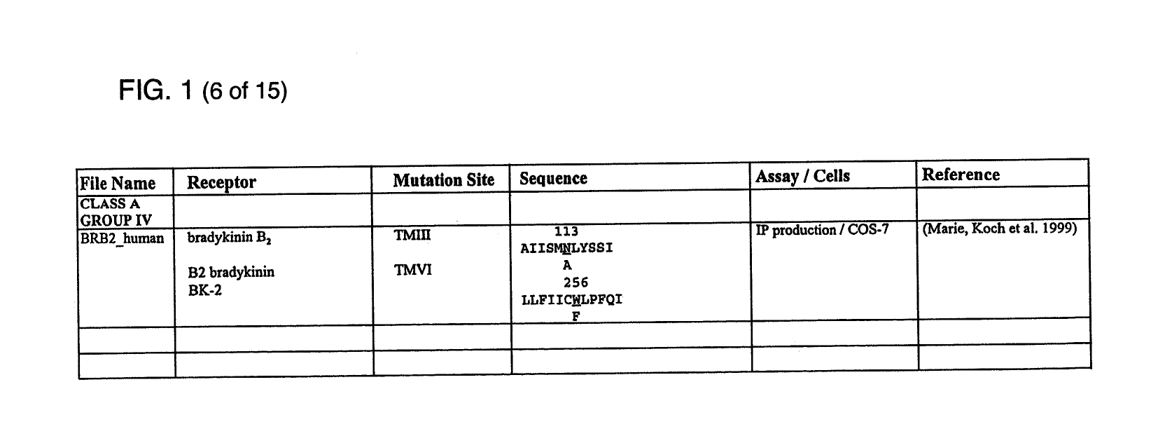

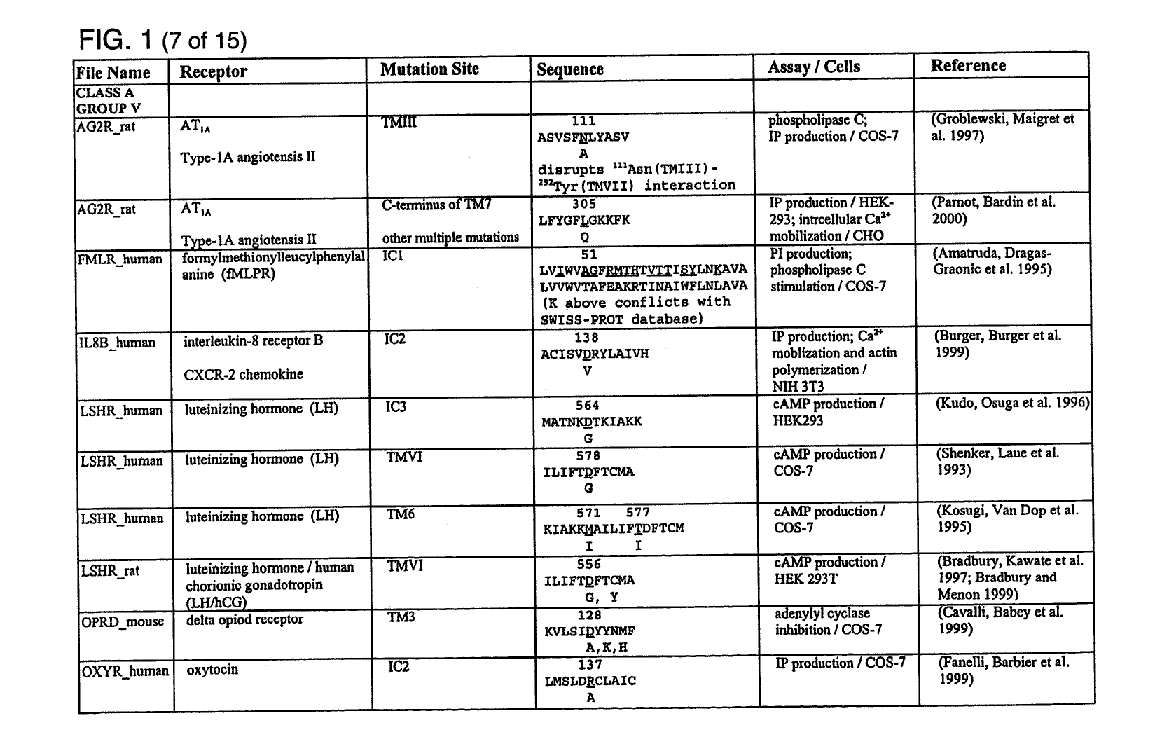

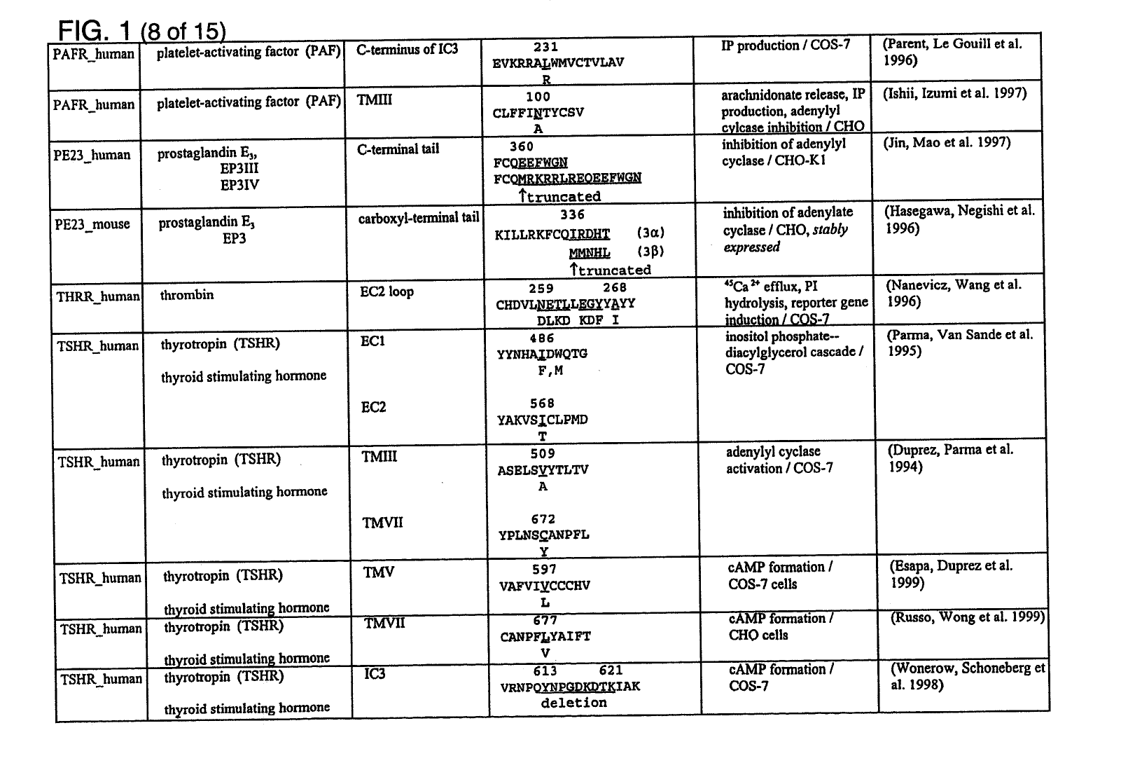

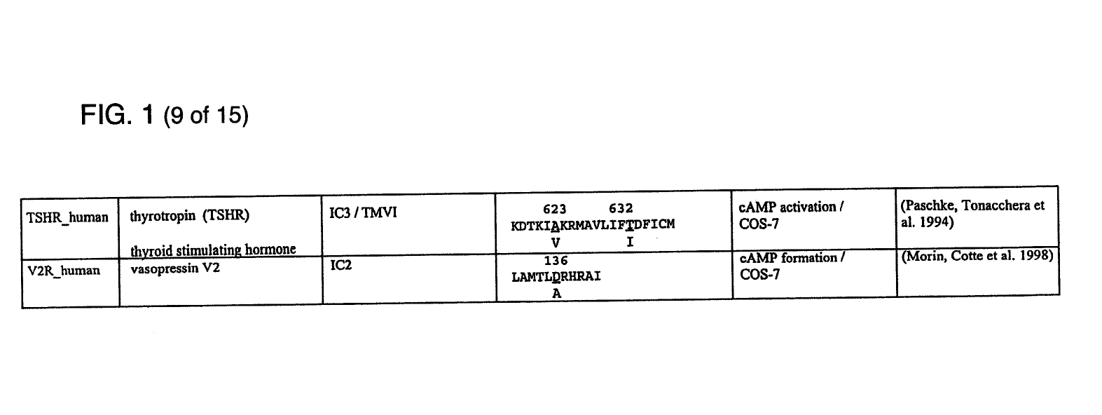

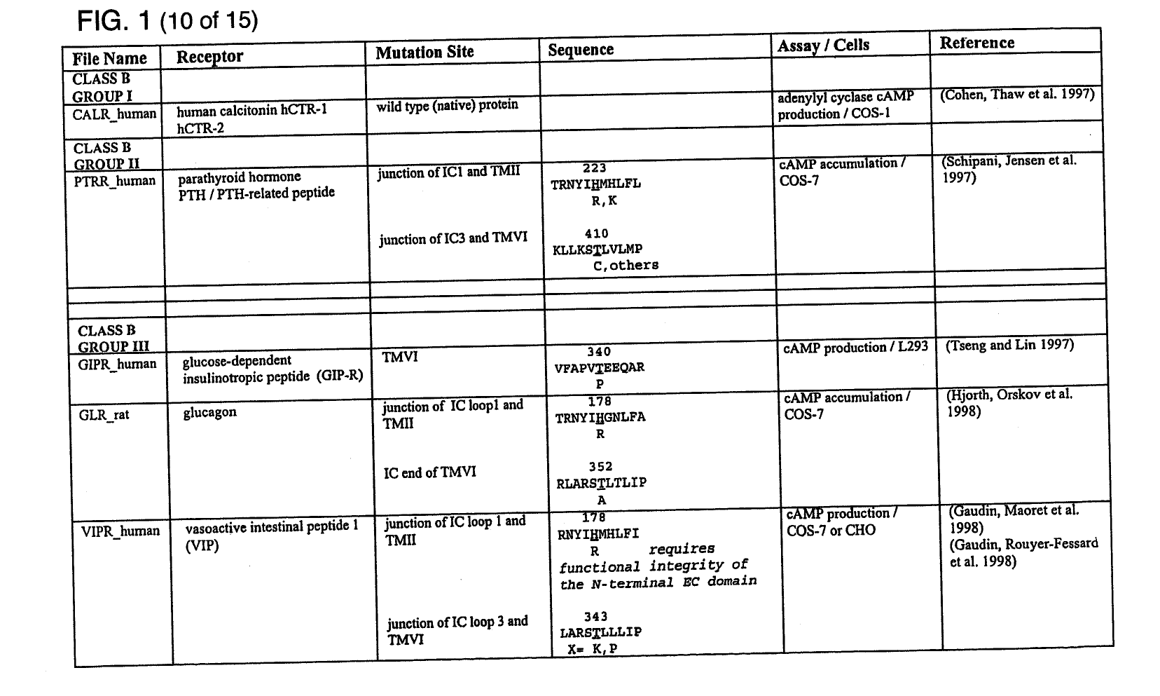

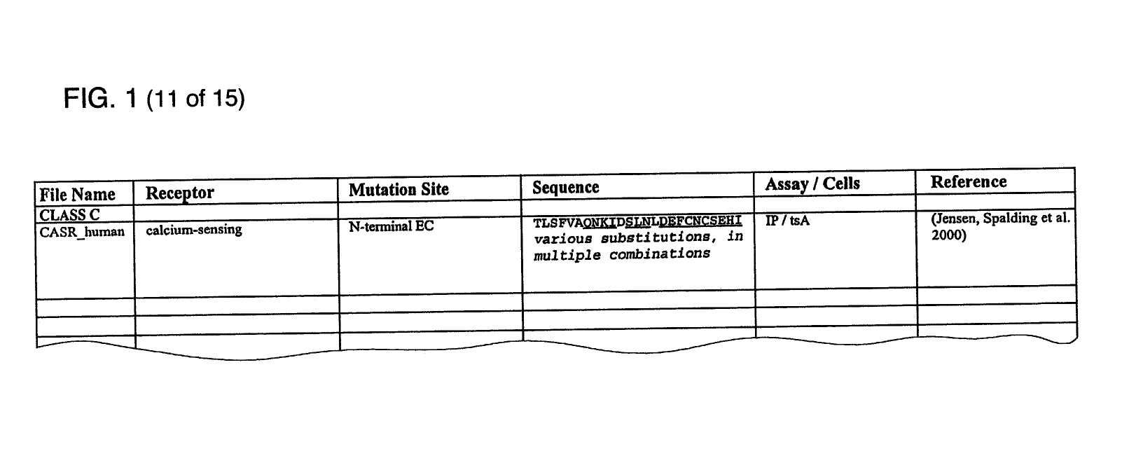

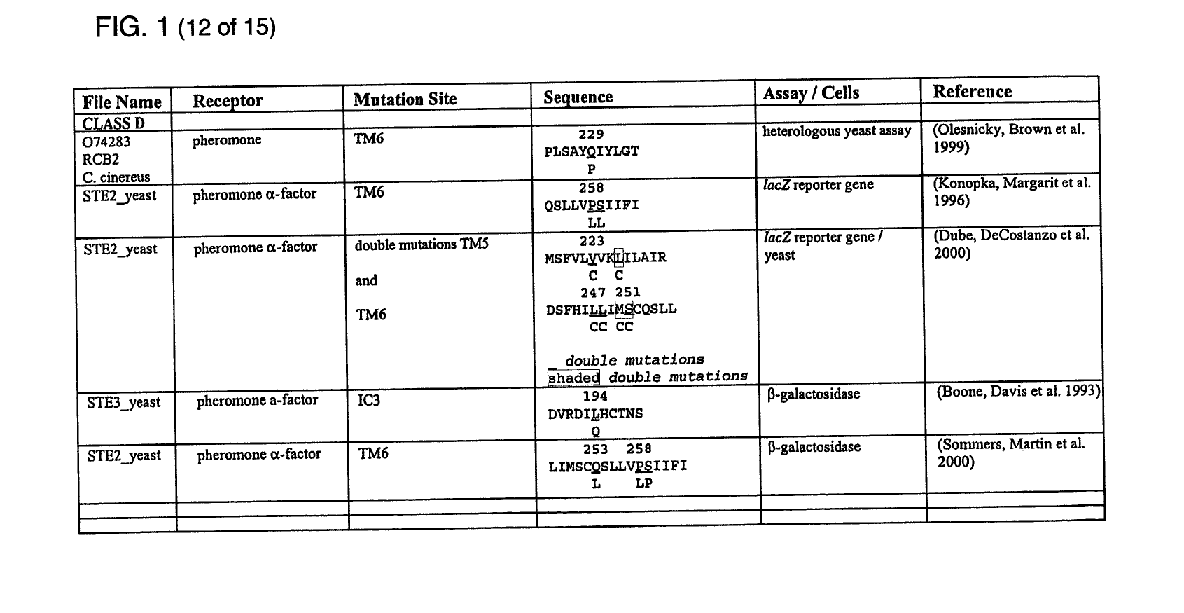

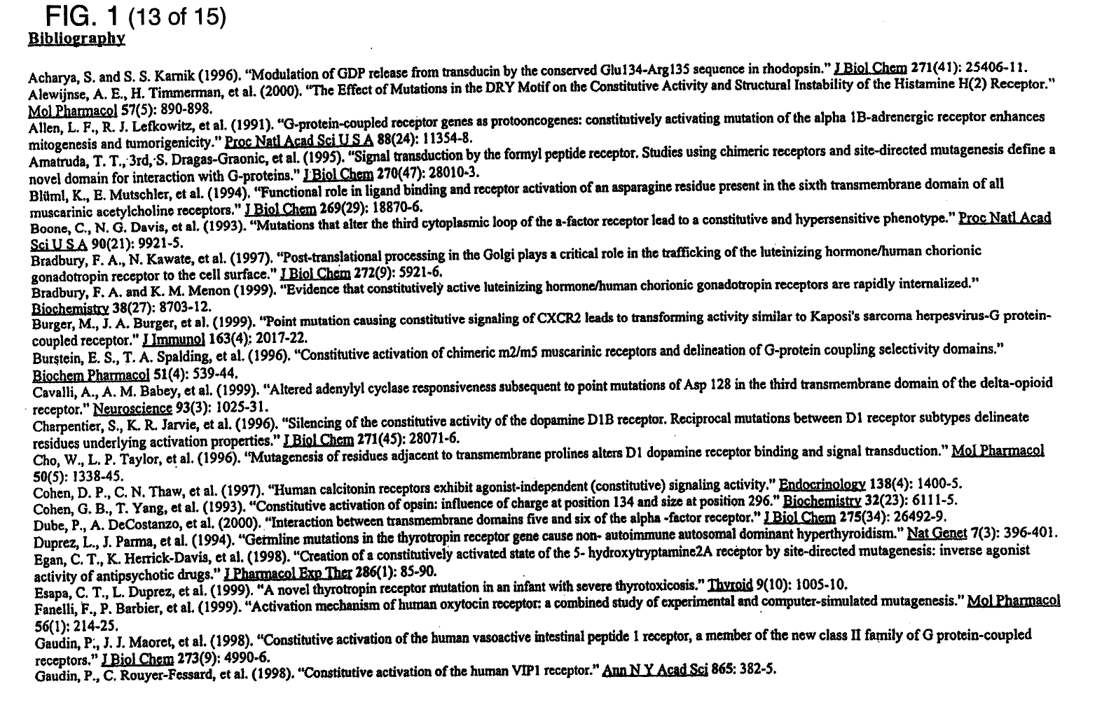

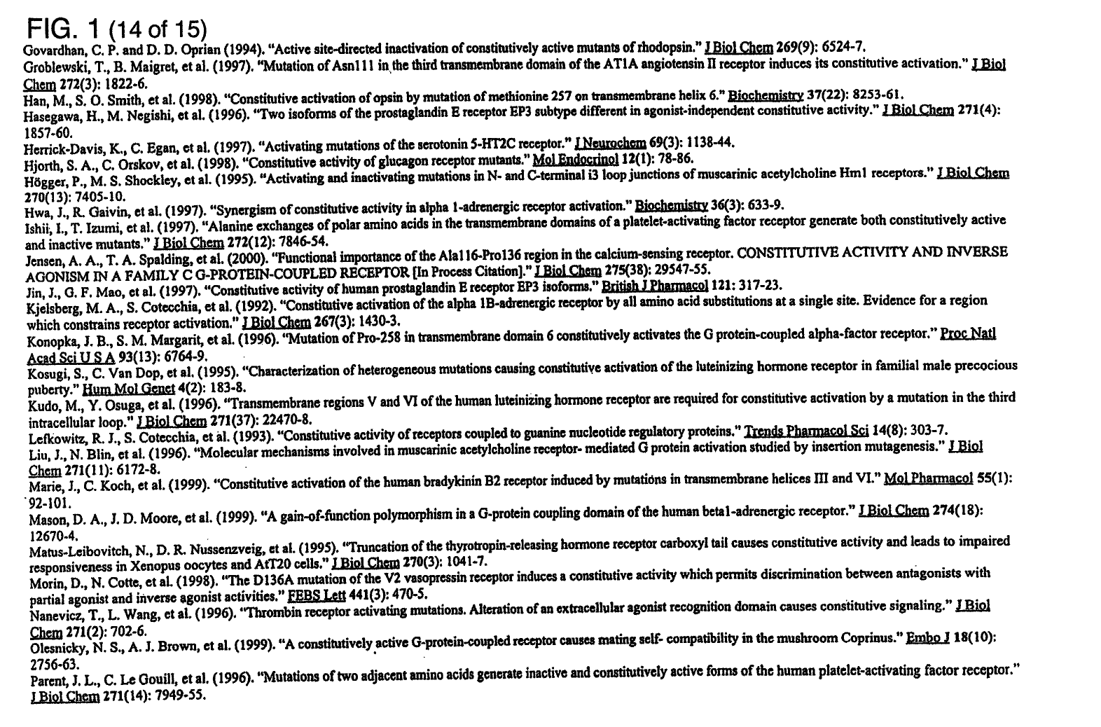

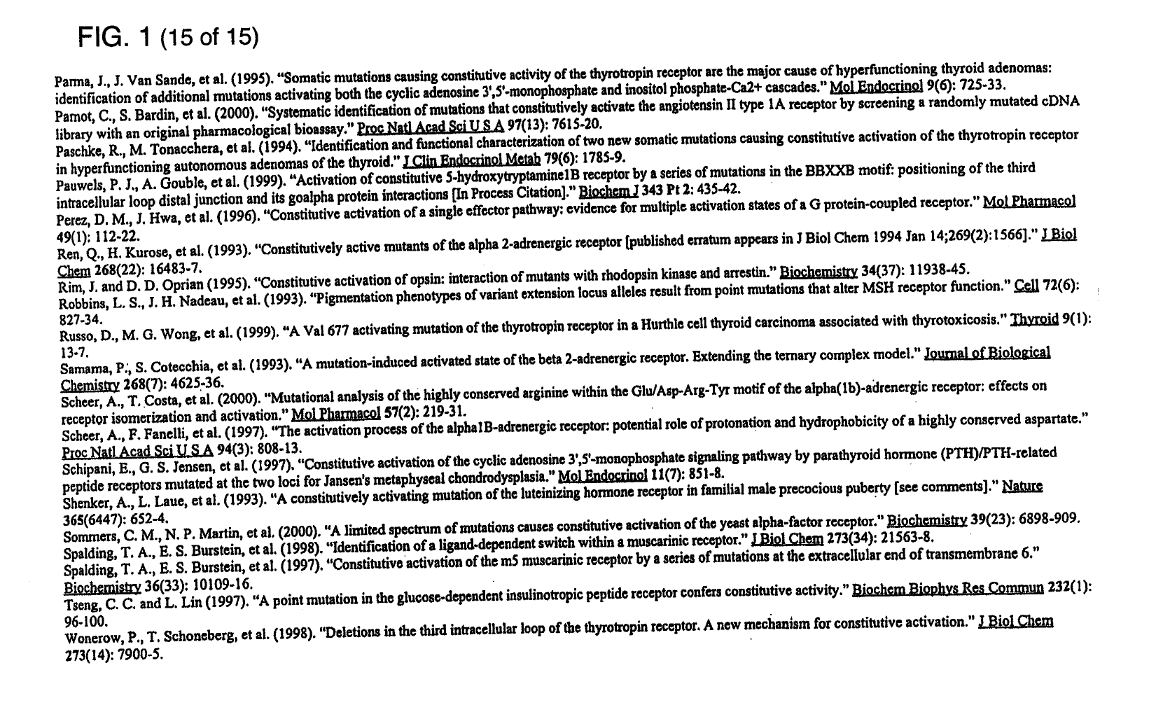

As will be appreciated by those skilled in the art, numerous constitutively active receptors (naturally occurring and non-naturally occurring) have been previously identified. Such receptors provide a wealth of information that can be used to identify additional constitutively active receptors. To complete step (1), above, available nucleic acid and/or amino acid sequence information, preferably amino acid sequence information, including wild-type and mutant receptors, is compiled to generate a database of constitutively active receptor sequences. Next, the sequence of a given nonconstitutively active receptor (including any orphan receptor) of therapeutic interest (e.g., a receptor known to be a receptor for an agonist) is compared to the many sequences of constitutively active receptors in the database to identify regions that are conserved between the nonconstitutively active receptor and the one or more constitutively active receptors. The present invention demonstrates step (1) by providing an extensive database of constitutively active Class I G protein-coupled receptors (see FIG. 1). One of ordinary skill in the art will appreciate that additional databases may easily be generated for other types of receptor molecules, for example, Class II G protein-coupled receptors (see Jüppner et al., Curr. Opin. Nephrol. Hypertens. 3(4):371-378, FIG. 1, p 373 (1994)). Databases may also be generated for polymorphic receptors.

In order to complete step (2), specific residues in the nonconstitutively active wild-type receptor are targeted for mutation based on the identified regions of homology between the nonconstitutively active receptor and constitutively active receptor(s), which are likely to impart constitutive activity onto the nonconstitutively active receptor. For example, if a region of homology between a nonconstitutively active receptor and a constitutively active receptor is identified that is identical in all amino acids but one, a mutation is introduced into the nonconstitutively active receptor to make the conserved region in the nonconstitutively active receptor identical to that of the constitutively active receptor. Alternatively, if the region conserved between the nonconstitutively active receptor and the constitutively active receptor shows a high degree of amino acid similarity, a series of targeted mutations are introduced into the nonconstitutively active receptor that are likely, based on the degree of homology and the knowledge of the skilled artisan, to make the receptor constitutively active. As but another example, the nonconstitutively active receptor might share a region of homology with another nonconstitutively active receptor that has been made constitutively active by the introduction of a certain mutation or mutations. In this case, the same or similar mutations are introduced into the given nonconstitutively active receptor.

Alternatively, the database is used to identify regions of homology between a naturally occurring receptor of therapeutic interest and one or more constitutively active receptors. The identified regions of homology would lead the skilled artisan to test the naturally occurring receptor for constitutive activity.

Applicants demonstrate step (2) by using the database of constitutively active Class I G protein-coupled receptors provided in step (1) (FIG. 1) to target specific residues in nonconstitutively active receptors for mutation. Briefly, highly conserved regions were identified between several nonconstitutively active receptors and a number of constitutively active Class I G protein-coupled receptors in the database. This information was used to target specific residues in the nonconstitutively active receptors for mutation. As described in detail below, targeted point mutations were introduced into the cholecystokinin-B/gastrin receptor (CCK-BR), melanocortin-4 (MC-4), and the mu opioid receptor, which imparted constitutive activity to the nonconstitutively active receptors (see Examples 1, 2, and 3). It will be appreciated that this method of comparing nonconstitutively active receptors and constitutively active receptors to identify regions of conservation may be repeated with any family of related receptors with the goal of targeting regions of homology for mutation, as set forth in steps (1) and (2) above.

Step (3) involves assaying the mutant receptors for constitutive activity by assaying for an increase in basal activity of the receptor. The present invention provides a reporter assay system in which a response element, responsive to signaling through a particular receptor, is attached to a reporter gene in combination with a transcriptional promoter. Specifically, the expression of the reporter gene is controlled by the activity of the chosen receptor. This method involves the steps of (1) identifying a response element that is sensitive to signaling by a specific receptor polypeptide (e.g., by eliciting an increase or decrease in gene expression upon receptor activation); (2) operably linking the response element and a promoter (if the promoter is not included in the response element) to a reporter gene; and (3) comparing the basal level reporter activity of a putative constitutively active receptor to a negative control, an increase in basal level reporter activity compared to the negative control indicating the identification of a constitutively active receptor. Preferably the increase in basal activity is at least two-fold, preferably three-fold, and most preferably at least six-fold over the basal activity of the negative control. In preferred embodiments, this assay system is used to screen for receptor mutants exhibiting constitutive activity.

It will be appreciated that the receptor can be any receptor identified as a candidate constitutively active receptor. In addition, one skilled in the art would recognize that the response element used in the present response assay can be any response element that is sensitive to signaling through the identified candidate constitutively active receptor. For example, in reporter assays for identifying constitutively active receptors that are coupled to different G proteins, one would select response elements that are sensitive to signaling through receptors coupled to G proteins. In particular examples, the somatostatin promoter element (SMS) is activated by coupling of receptors to either Gαq or Gαs; the serum response element (SRE) is activated by receptor coupling to Gαq; the cAMP response element (CRE) is activated by receptor coupling to Gαs and inhibited by coupling to Gαi; and the TPA response element (sensitive to phorbol esters) is activated by receptor coupling to Gαq. Each of these response elements can be employed in a reporter assay to generate a readout for the basal level activity of a specific G protein-coupled receptor.

In addition, a reporter construct for detecting receptor signaling might include a response element that is a promoter sensitive to signaling through a particular receptor. For example, the promoters of genes encoding epidermal growth factor, gastrin, or fos can be operably linked to a reporter gene for detection of G protein-coupled receptor signaling.

It will be appreciated that a wide variety of reporter constructs can be generated that are sensitive to any of a variety of signaling pathways induced by signaling through a particular receptor (e.g., a second messenger signaling pathway). Accordingly, this assay system may be used to identify other types of constitutively active receptors, including receptors that are single transmembrane receptors or nuclear receptors, by simply selecting a response element that is sensitive to the particular receptor and positioning the response element upstream of a reporter gene in a reporter construct. For example, the elements AP-1, NF-κb, SRF, MAP kinase, p53, c-jun, TARE can all be positioned upstream of a reporter gene to obtain reporter gene expression. Additional response elements, including promoter elements, can be found in the Stratagene catalog (PathDetect® in Vivo Signal Transduction Pathway cis-Reporting Systems Introduction Manual or PathDetect® in Vivo Signal Transduction Pathway trans-Reporting Systems Introduction Manual, Stratagene, La Jolla, Calif.).

In one preferred embodiment, the present invention provides a G protein-coupled reporter assay system including (1) a reporter construct containing a response element that is sensitive to signaling through a specific G protein, and a promoter, operably linked to a reporter gene; preferably in combination with (2) an expression vector containing a promoter operably linked to a nucleic acid encoding a receptor, wherein the receptor is coupled to a G protein, or other downstream mediator, to which the selected response element is sensitive.

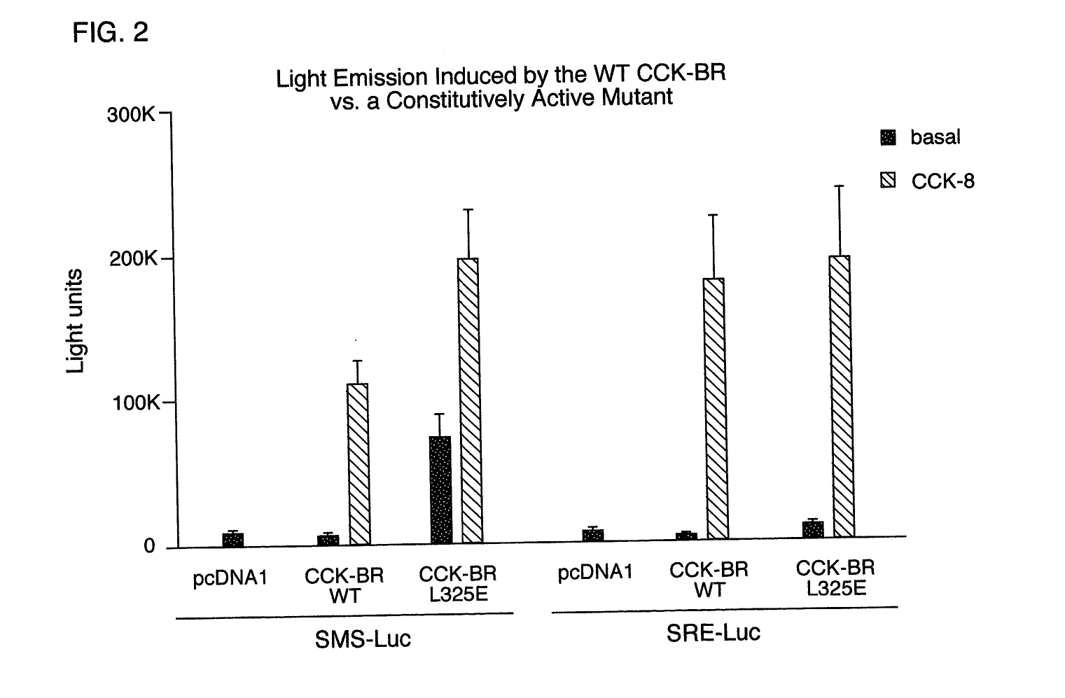

The present invention demonstrates use of specific response elements that are sensitive to signaling through each of Gαq, Gαs, and Gαi. For example, the SMS and SRE response elements each detect an increase in basal activity of the Leu325Glu CCK-BR mutant receptor, which is coupled to Gαq (see FIG. 2).

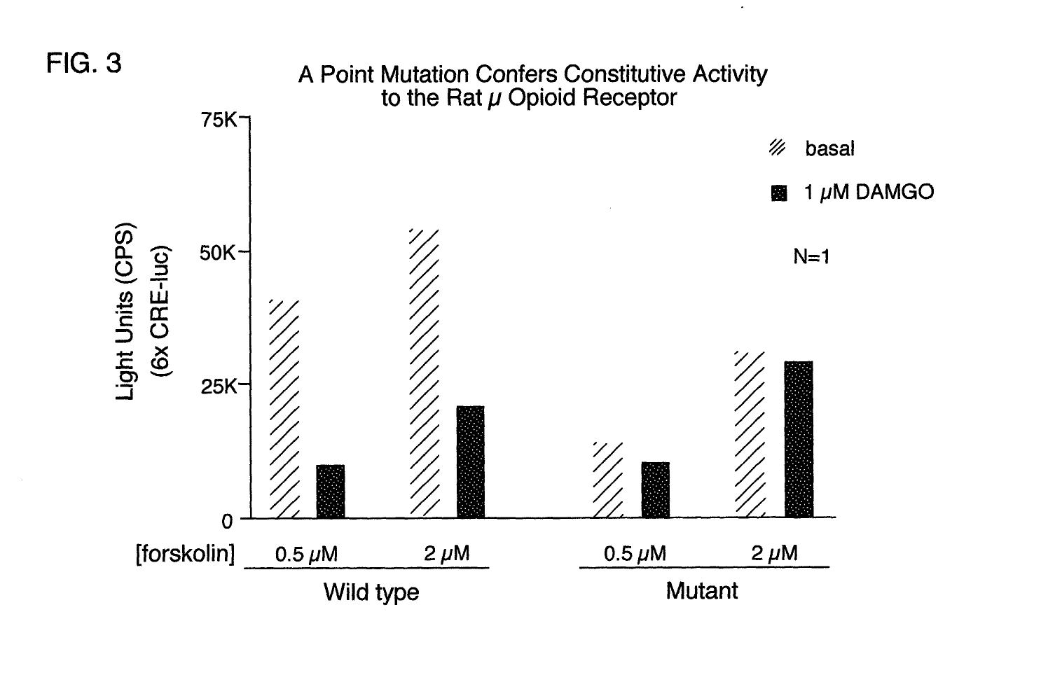

Similarly, a constitutively active rat mu opioid receptor was identified using a reporter construct sensitive to Gαi coupling (see FIG. 3). The response element employed in this assay was the cAMP-response element (CRE), which is sensitive to Gαi mediated changes in intracellular levels of cAMP. Signaling through the rat mu opioid receptor via Gαi inhibits adenylate cyclase, causing a decrease in intracellular cAMP. Therefore, an increase in rat mu opioid receptor signaling induces a decrease in CRE mediated reporter activity.

Prior to the present invention, Gαi-mediated decreases in intracellular cAMP were measured by (1) stimulating cells with forskolin, which causes receptor-independent activation of adenylate cyclase and generates an intracellular pool of cAMP; (2) stimulating the cells with ligand; and (3) measuring the ligand-induced, receptor-dependent Gαi-mediated decrease in the intracellular cAMP pool (e.g., using a radioimmunoassay (e.g., New England Nuclear, Boston, Mass.)). As demonstrated herein, the approach of the present invention was capable of identifying a constitutively active rat mu opioid receptor (FIG. 3). Specifically, cells transfected with a CRE-Luc reporter construct (Stratagene, La Jolla, Calif.) and an expression vector encoding either a wild-type or a mutant rat mu opioid receptor were stimulated with 0.5 μM or 2 μM forskolin to increase the intracellular pool of cAMP. The basal (and ligand-induced) level of receptor activity was then measured using a standard luciferase assay (see FIG. 3). Coexpression of the receptor of interest with a luciferase reporter gene construct allows one to measure light emission as a readout for basal signaling.

The results illustrated in FIG. 3 show a reduction in basal activity in the mutant rat mu opioid receptor compared to the wild-type rat mu opioid receptor. This decrease in activity indicates an increase in the basal level activity of the mutant rat mu opioid receptor, because activation of the rat mu opioid receptor induces a decrease in CRE-mediated reporter activity (FIG. 3, compare 0.5. μM wild-type to 0.5 μM mutant). It is important to note that the level of constitutive activity in the mutant rat mu opioid receptor approximates the level of ligand-stimulated activity of the wild-type receptor.

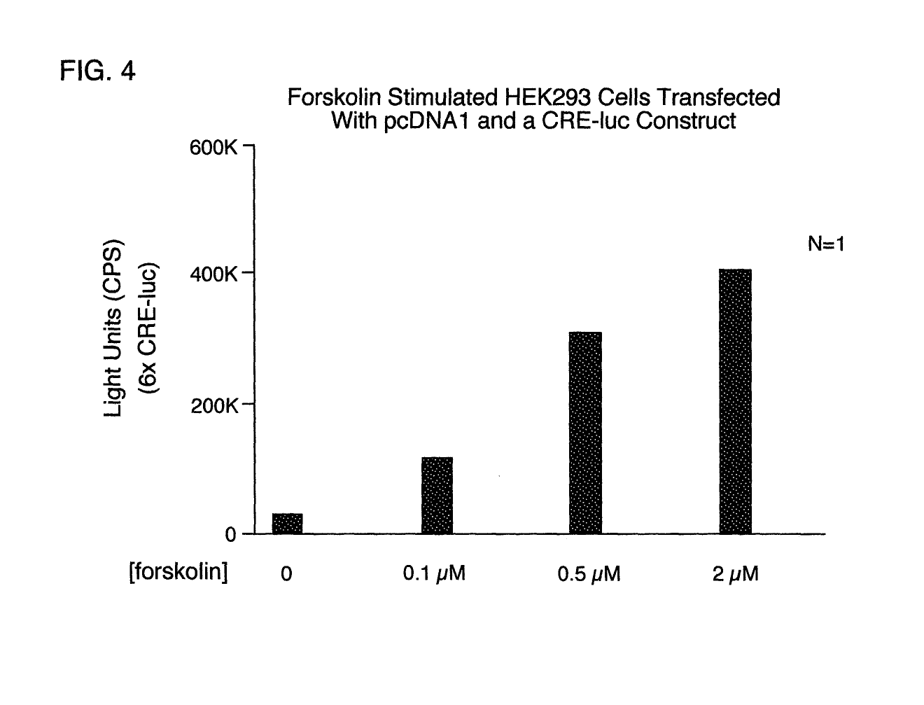

Although successful, use of the inventive assay to measure Gαi coupling directly has several disadvantages. First, detecting Gαi-mediated inhibition of cAMP requires overcoming the simultaneous positive effects of forskolin on adenylate cyclase. For example, FIG. 4 illustrates the positive effect of forskolin in HEK293 cells on the response of CRE-Luc in the absence of a contransfected receptor protein. In addition, detection of a ligand-stimulated decrease in intracellular cAMP relies on whether a large enough percentage of the cells are successfully transfected with, and express, the receptor molecule. Moreover, when using transient transfection assays, instead of stably transfected cell lines, interexperimental variation occurs because the percentage of cells transfected from one experiment to the next is difficult to control.

A positive assay for Gαi coupling (i.e., an assay that yields an increase in luciferase activity upon receptor activation, instead of a negative assay that yields a decrease in luciferase activity upon receptor activation), provides a more detectable output signal and less interassay variation. It was hypothesized that Gαi coupling could be detected by altering the signaling pathway generated by Gαi coupled receptors. A chimeric G protein (Gq5i), Broach and Thorner, Nature 384 (Suppl.): 14-16 (1996), that contains the entire Gαq protein having the five C-terminal amino acids from Gαi attached to the C-terminus of Gαq has been generated. This chimeric G protein is recognized as Gαi by Gαi coupled receptors, but switches the receptor induced signaling from Gαi to Gαq. This allows Gαi receptor coupling to be detected using a positive assay by use of the Gαq responsive SMS-Luc or SRE-Luc construct (Stratagene, La Jolla, Calif.). SMS and SRE preferably respond to Gαq mediated inositol and calcium production. Moreover, detection can be carried out in the absence of forskolin pre-stimulation of cells.

Other chimeric G proteins that can be used according to the methods of the invention include those shown in Appendix 1 (G Protein Users Manual, http://gweb1.ucsf.edu/labs/Conklin/technical/GproteinManual.html) and described in Milligan, G. and S. Rees, TIPS 20:118-124, 1999, and Conklin et al., Nature 363: 274-276, 1993, incorporated by reference herein. Moreover, any other chimeric G protein can be constructed by replacing or adding at least 3 amino acids, usually at least 5 amino acids, from the carboxyl terminus of a G protein (e.g., Gi, Gq, Gs, Gz, or Go) to a second G protein (e.g., Gi, Gq, Gs, Gz, or Go) which is either full-length or includes at least 50% of the amino terminal amino acids.

Generally, the carboxyl-terminus of the G alpha protein subunit is a key determinant of receptor specificity. For example, the Gq alpha subunit (alpha q) can be made to respond to Gi alpha-coupled receptors by replacing its carboxyl-terminus with the corresponding Gi2 alpha, Go alpha, or Gz alpha residues. In addition, C-terminal mutations of Gq alpha/Gi alpha chimeras show that the critical amino acids are in the -3 and -4 positions, and exchange of carboxyl-termini between Gq alpha and Gs alpha allows activation by receptors appropriate to the C-terminal residues. Furthermore, replacement of the five carboxyl-terminal amino acids of Gq alpha with the Gs alpha sequence permitted a certain Gs alpha-coupled receptor (the V2 vasopressin receptor, but not the beta 2-adrenoceptor) to stimulate phospholipase C. Replacement of the five carboxyl-terminal amino acids of Gs alpha with residues of Gq alpha permitted certain Gq alpha-coupled receptors (bombesin and V1a vasopressin receptors, but not the Oxytocin receptor) to stimulate adenylyl cyclase. Thus, the relative importance of the G alpha carboxyl-terminus for permitting coupling to a new receptor depends on the receptor with which it is paired.

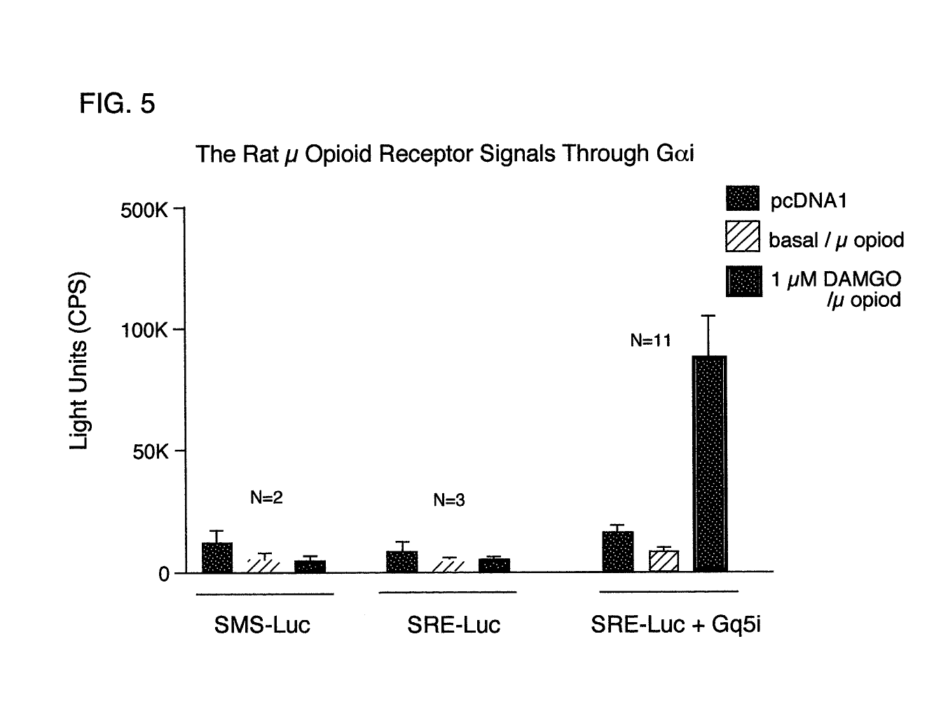

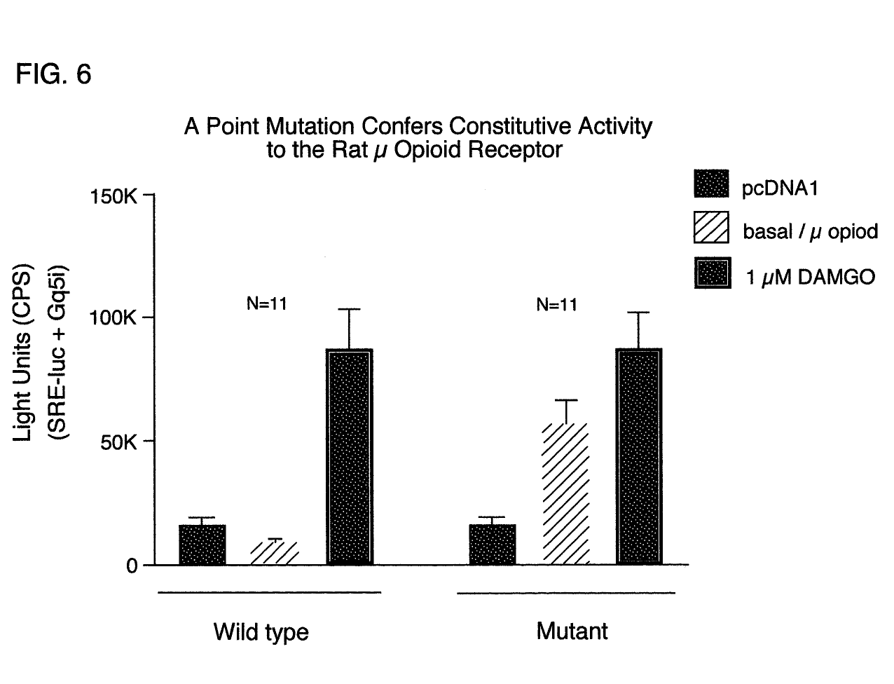

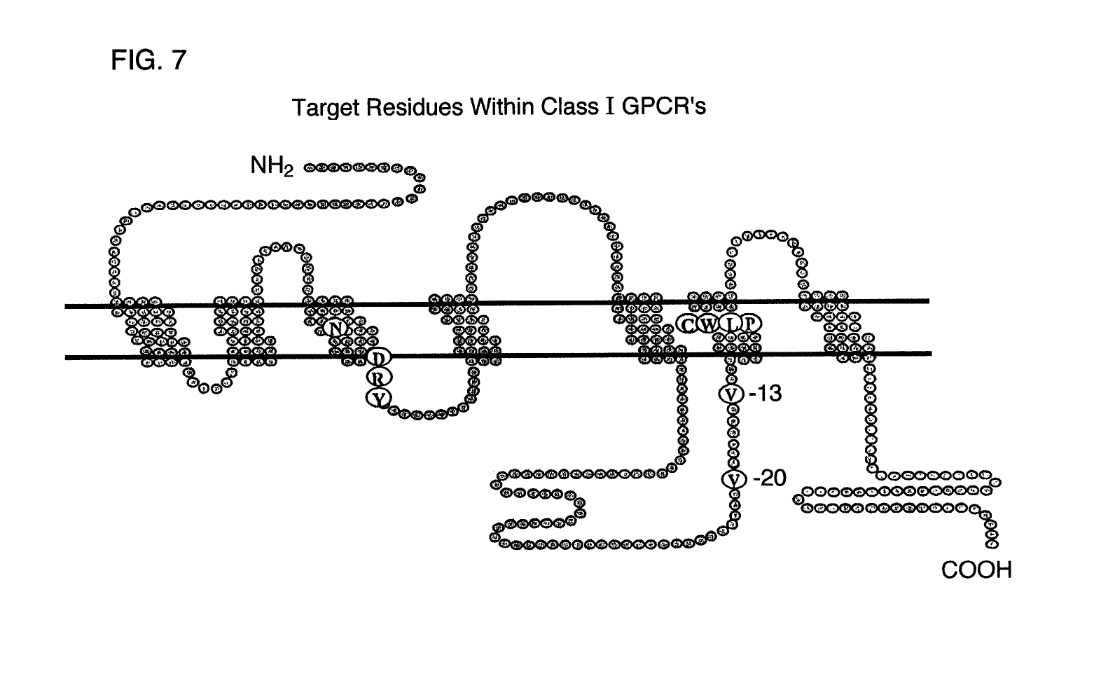

As demonstrated in FIG. 5, Gq5i can be used to detect rat mu opioid receptor coupling to Gαi. FIG. 5 shows that ligand-stimulated luciferase activity is not detected in response to ligand stimulation using luciferase constructs having either the SMS or SRE alone (left two columns), whereas a large increase in ligand-stimulated luciferase activity is detected using SRE-Luc in combination with Gq5i (far right). This assay was also employed to measure the constitutive activity of the Asn150Ala mutant rat mu opioid receptor (FIG. 6).

Any other G protein chimera that is capable of switching the signaling from one G-protein coupled receptor to another pathway can also be used according to the invention.

### Applications

In one preferred embodiment, the constitutively active receptors identified by the screening assays of the present invention are used as tools for identifying the ligand of a given receptor, including peptide, non-peptide, and small molecule ligands. For example, ligands (e.g., a hormone or a drug) that bind a particular constitutively active receptor may be identified using a reporter assay system by (1) operably linking a response element, which is sensitive to receptor activation, and a promoter, to a reporter gene to generate a receptor activation sensitive reporter construct; (2) cotransfecting cells with the reporter construct and an expression vector containing nucleic acid encoding the constitutively active receptor; (3) contacting the cells with a ligand; and 4) assaying for ligand-dependent activation or inhibition of the reporter construct, an increase or decrease in the ligand-dependent activation, compared ligand-independent signaling, indicating the presence of an agonist or inverse agonist, respectfully. Ligands that activate or inhibit a particular receptor by increasing or decreasing receptor activity may, upon further experimentation, prove to be valuable therapeutic drugs for treatment of disease.

In yet another preferred embodiment, the assay systems of the present invention may be used to screen for genetic polymorphisms or mutations that alter (i.e., increase or decrease) the basal or ligand-stimulated signal generated by a particular receptor. In one particularly preferred embodiment, the identified polymorphisms or mutations result in agonist independent signaling, particularly agonist independent signaling that may cause disease. Alternatively, the identified polymorphisms or mutations result in an altered response to a drug. In another preferred embodiment, the assay systems of the present invention can be used to detect mutation-induced sensitivity of a receptor to ligand binding (e.g., by identifying a hypersensitive receptor). With the emergence of pharmacogenomics, rapid methods of screening for functionally important polymorphisms or mutations are highly valuable. Indeed, any mutant or polymorphic receptor can be placed in an expression vector and used in the assay systems of the present invention.

In another preferred embodiment, when applied to constitutively active orphan receptors (wild-type or mutant), a panel of reporter gene constructs that are sensitive to different signaling pathways (e.g., SRE-Luc, SMS-Luc, and CRE-Luc) can be used to predict the second messenger pathway that will be activated by the endogenous receptor ligand (e.g., cAMP, inositol phosphate production). This information will facilitate and accelerate both the identification of cognate endogenous ligands (i.e., the de-orphaning of a receptor), and the discovery of drugs that act on orphan receptors by the use of the inventive high-throughput screening based techniques.

In a related embodiment, the present invention provides a novel assay system for identifying the G protein to which a particular receptor is coupled in the form of reporter constructs responsive to Gαq, Gαs, or Gαi-mediated signaling. In one preferred embodiment, the present invention provides a panel of reporter constructs that are capable of determining which G protein a particular receptor is coupled to, selected from Gαq, Gαs, and Gαi. The assay system requires (1) a panel of reporter constructs containing a response element sensitive to a particular G protein, selected from Gαq, Gαs, and Gαi, and a promoter operably linked to a reporter gene; (2) an expression vector encoding a G protein-coupled receptor; and (3) a cell into which to deliver the components of (1) and (2).

In another preferred embodiment, the present invention provides a method of identifying the G protein to which a receptor is coupled, comprising the steps of: (1) selecting a G protein-coupled receptor; (2) using an expression vector encoding the selected G protein-coupled receptor in combination with a panel of reporter assays that are capable of detecting coupling to Gαq, Gαs, or Gαi (as described above); and (3) comparing the signal generated by each assay in response to ligand stimulation, an increase in reporter activity in one reporter assay, and not the other two, indicating coupling to the G protein to which the reporter assay is sensitive. Some particularly preferred response elements include SMS, SRE, and CRE. In certain preferred embodiments, the reporter assay further includes a chimeric G protein capable of switching the signaling of the receptor to a different pathway than the wild-type receptor. Preferably this signaling pathway generates a positive signal in the reporter assay, as opposed to a negative signal. One particularly preferred chimeric G protein is the chimeric G protein, Gq5i (Broach and Thorner, supra), described above.

### Mu Opioid Receptor

According to the present invention, nucleic acids are identified that encode clinically useful constitutively active receptors. We demonstrate this aspect of the invention by identifying a constitutively active mu opioid receptor.

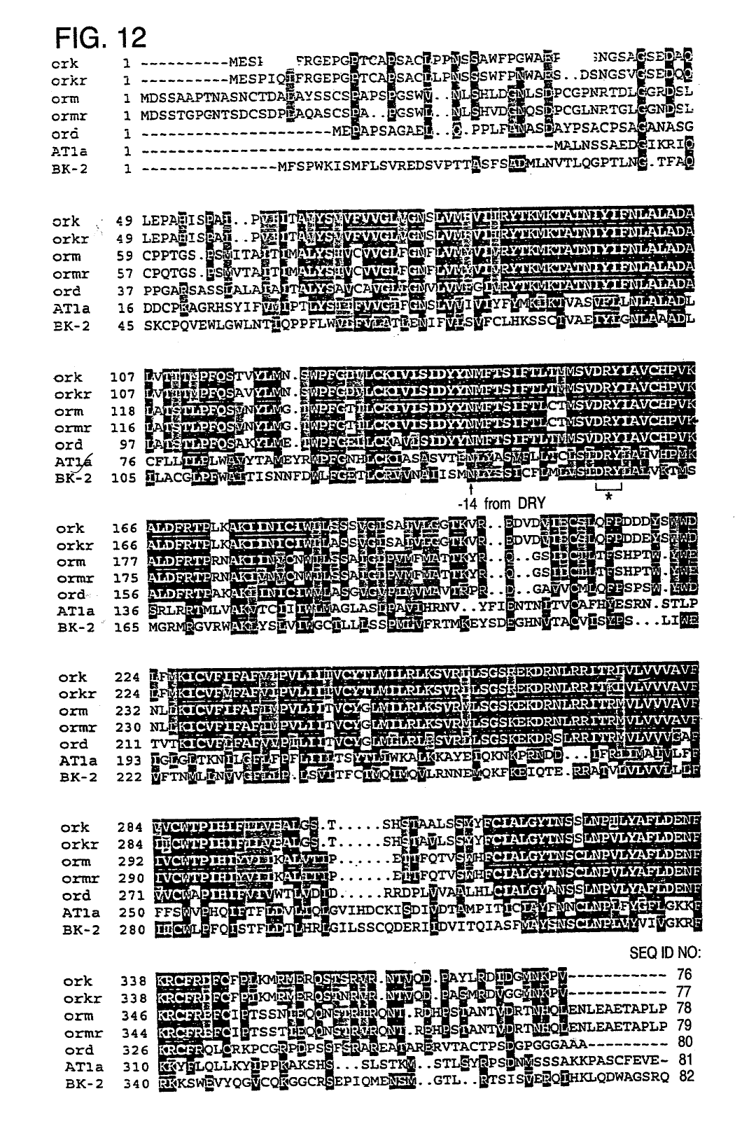

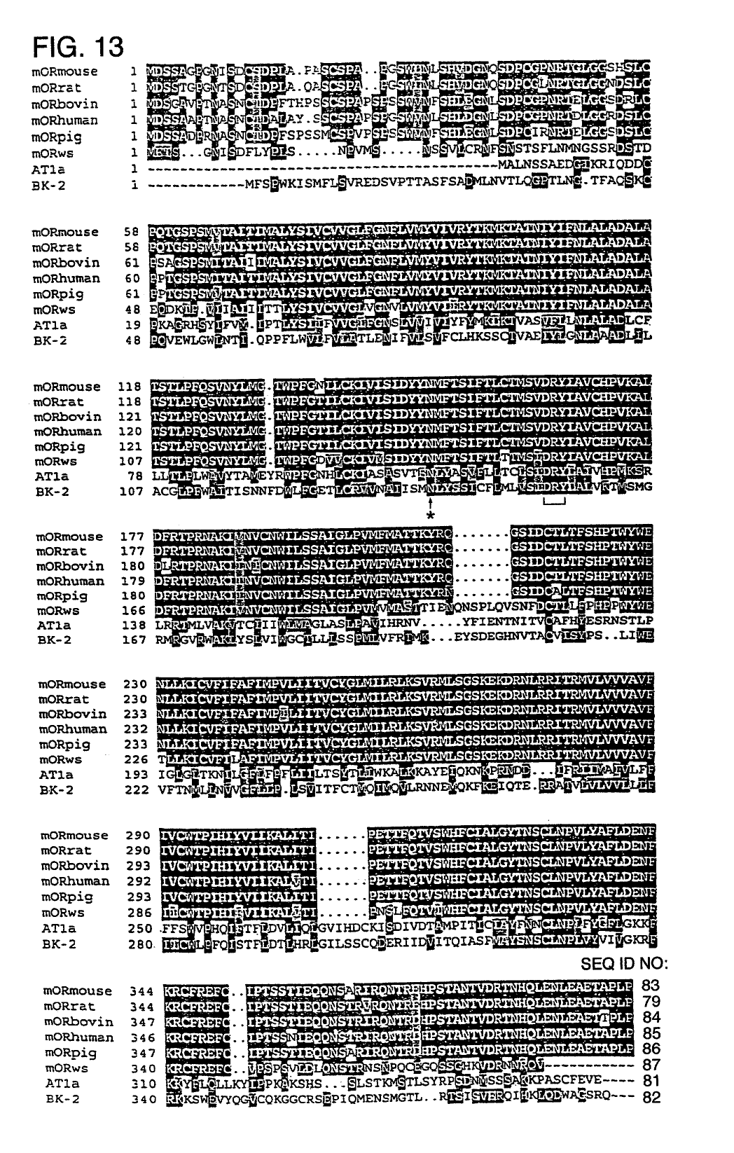

The mu opioid receptor is an opiate receptor that falls within the G protein-linked seven transmembrane domain neuropeptide receptor family. In general, opiate receptors (including μ(mu), κ, δ, and opiate-like receptor (OLR)) couple to guanine nucleotide binding (G) proteins (Li et al. supra) (see FIGS. 12 and 13). For example, opiates can alter GTP hydrolysis, GTP analogs and pertussis toxin can change opiate receptor binding, and opiates can influence G-protein-linked second messenger systems and ion channels. More specifically, mu opioid receptors have a characteristic high affinity for morphine and other opiate drugs and peptides. Binding of morphine to the mu opioid receptor results in an analgesic and euphoric effect, common to opiate drugs.

A single point mutation (Asn to Ala at amino acid 150) was introduced into the third transmembrane region of the rat mu opioid receptor (SEQ ID NO: 1). This Asn residue was targeted for mutation based on it being highly conserved between the mu opioid receptor, the bradykinin B2 receptor, and the angiotensin II AT1A receptor. Furthermore, homologous mutations at this residue in the bradykinin B2 and angiotensin II AT1A receptors yielded receptors having constitutive activity. Indeed, the Asn150Ala mu opioid receptor mutant exhibited levels of basal activity which exceeded 50% of the maximal level of ligand-stimulated second messenger signaling (see Example 1).

### EXAMPLES

The present invention can be further understood through consideration of the following non-limiting examples.

### Example 1

### Constitutively Active Mu Opioid Receptor

This example describes the identification of a novel constitutively active rat mu opioid receptor.

### Identifying Regions of Homology in the Mu Opioid Receptor

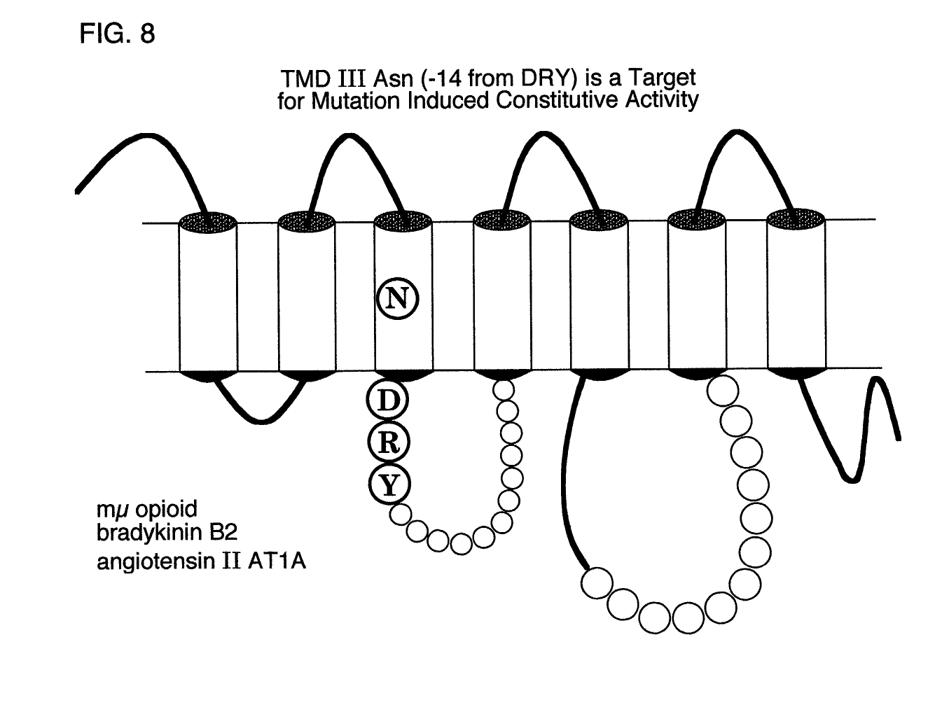

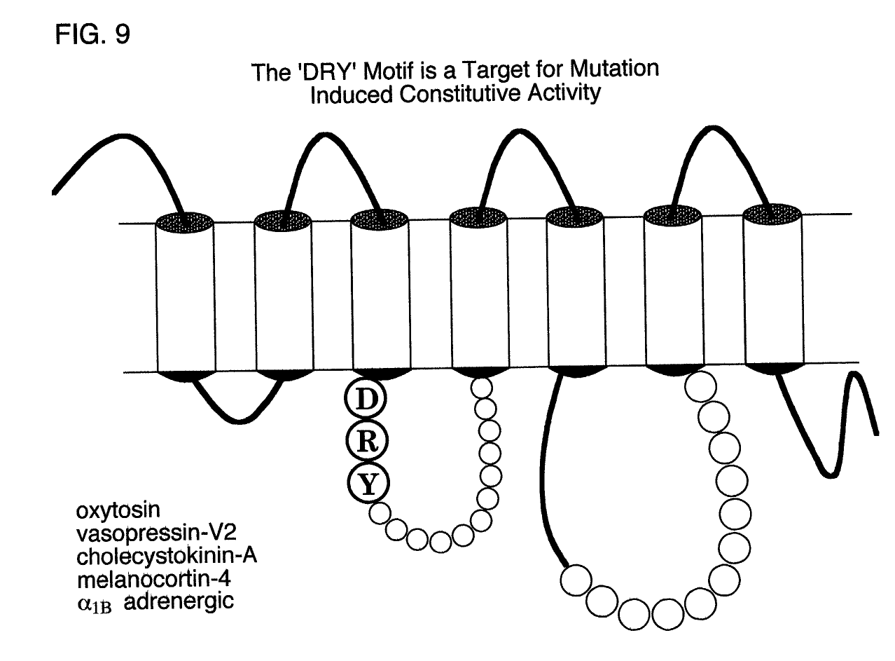

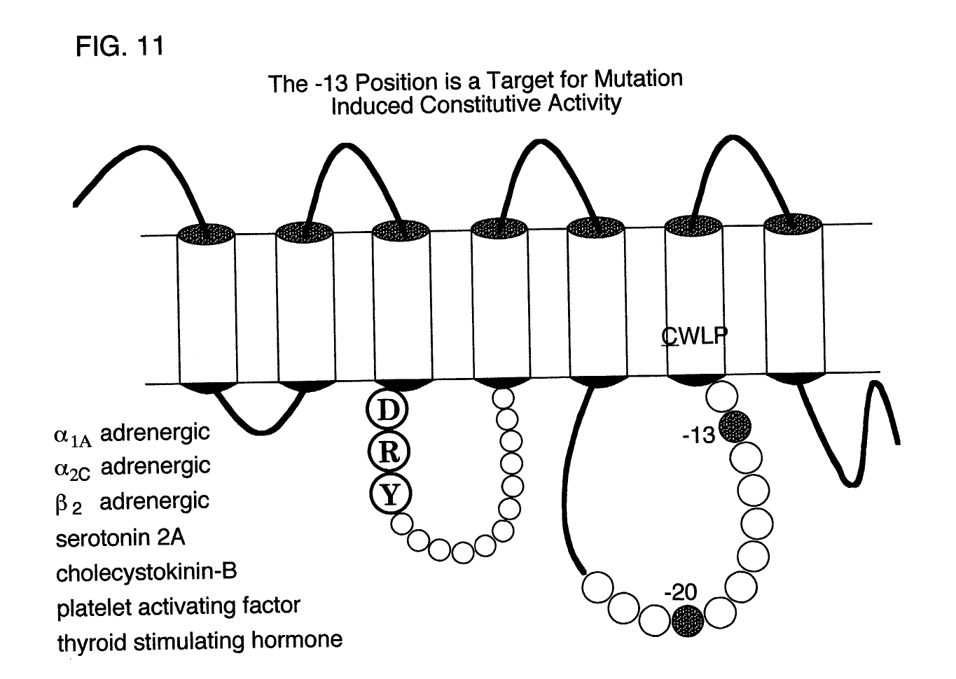

A database containing sequence information for known constitutively active Class I G protein-coupled receptors was generated by compiling available information from the prior art (see FIG. 1). The database was then used to identify key residues within Class I G protein-coupled receptors that are important for constitutive activity. These highly conserved residues are illustrated in FIG. 8. Of particular interest was the Asn residue at position 150 of SEQ ID NO: 1 in transmembrane domain III, which is conserved between the rat mu opioid receptor, the bradykinin B2 receptor, and the angiotensin II AT1A receptor (see FIG. 8). The ‘DRY’ motif at position 164-166 of SEQ ID NO: 1 is conserved between the oxytocin receptor, the vasopressin-V2 receptor, the cholecystokinin-A (CCK-A) receptor, the melanocortin-4 (MC-4) receptor, and the α_{1B} adrenergic receptor (see FIG. 9). In addition, positions corresponding to 13 and 20 residues N-terminal to the CWLP motif are conserved between the 1A adrenergic receptor, the α2C adrenergic receptor, the β2 adrenergic receptor, the CCK-B receptor, the platelet activating factor receptor, and the thyroid stimulating hormone receptor (see FIG. 11).

### Generating Mutant Mu Opioid Receptors

Based on the homology between the mu opioid receptor, the bradykinin B2, and the angiotensin II AT1A receptors at the Asn residue at position 150 of SEQ ID NO: 1, we chose to generate a rat mu opioid receptor having a point mutation at this position. An Asn150Ala mutation was introduced into the rat mu opioid receptor using standard molecular biological techniques. This mutant gene was then subcloned into expression vector pcDNA1 (Sambrook et al. supra).

### Assaying Mutant Mu Opioid Receptors for Constitutive Activity

Reagents & Solutions: The cell culture media used in the assays described below was Gibco BRL #12100-046. This media was made according to manufacturer's recipe, pH adjusted to 7.2, filtered (0.22 micron pore), and supplemented with 1% Pen/Strep (Gibco #15140-122; 100% penicillin G 10,000 units/ml, and streptomycin 10,000 μg/ml) and 10% fetal bovine serum. Cell culture media lacking 10% fetal bovine serum was also generated. DNA used in the transfection experiments was purified and quantitated by measuring the absorbance at OD260. A LucLite Luciferase Assay Kit (Packard) was used to quantitate luciferase activity. Transfections were carried out using LipofectAMINE Reagent (Gibco #18324-012).

Constitutive activity of the Asn150Ala mutant rat mu opioid receptor was assessed using a luciferase assay. The rat mu opioid receptor is a Gαi coupled receptor. Therefore we chose to use the Gq5i reporter system, described in detail above (Broach and Thorner, supra), which switches the signaling pathway from Gαi to Gαq for reliable positive readout. HEK293 cells were transfected with the reporter construct SRE-Luc, an expression vector containing nucleic acid encoding Gq5i (Broach and Thorner, supra), and an expression vector containing nucleic acid encoding either the wild-type or the Asn150Ala mutant rat mu opioid receptor. Basal and ligand-stimulated luciferase activity was measured. The ligand used in this assay was [D-Ala²-MePhe⁴, Gly-ol⁵]enkephalin] (DAMGO). As a negative control, HEK293 cells were transfected with pcDNA1 (empty vector DNA), SRE-Luc, and the expression vector containing nucleic acid encoding Gq5i (Broach and Thorner, supra).

The luciferase assay was carried out as follows. On day 1, HEK293 cells in a T75 flask were washed with 15 ml serum-free media (or PBS), trypsinized with 5 ml 0.05% trypsin-EDTA (Gibco #25300-062), incubated at 37° C. for 3 minutes at which time 6-7 ml complete HEK293 media (Gibco #12100-046) and 10% Fetal Bovine Serum (Intergen #1050-90) were added. Thereafter, cells were collected in 50 ml centrifuge tubes, pelleted at 800-900 rpm (RCF ˜275), and resuspend in 20 ml complete media. The cells were counted using a haemocytometer and diluted to 85,000 cells/ml in complete media. Using a repeat pipettor or cell plater, 100 μl of cells were added to each well of a Primaria 96-well plate (Falcon #353872). Cells were then incubated at 37° C., 5% CO₂ until use at 48 hours.

On day 3, cells were transfected using LipofectAMINE™ according to the manufacturer's protocol (Gibco #18324-012, Rockville, Md.).

On day 4, cells were stimulated as follows. Ligands for the receptor, either DAMGO or a non-peptide ligand (e.g., naltrexene or nalonin), were diluted to a desired concentration in serum-free media containing 0.15 mM PMSF (or other protease inhibitor(s)). The transfection media was then completely removed from cells and 50-100 μl stimulation media (i.e., media containing candidate ligands or the corresponding ligand free solvent) was added to each well. The cells were incubated for the desired time (standard is overnight) at 37° C., 5% CO₂, although the optimal stimulation time may vary depending on the particular receptor used. The optimal incubation time may be determined systematically by testing a range of incubation times and determining which one yields the highest level of stimulation. For concomitant assessment of two ligands (e.g., ligand induced inhibition of forskolin stimulated CRE activity) each stimulus is prepared at two times the desired final concentration and mixed in equal volumes prior to addition to cells.

On day 5, an assay for luciferase expression was carried out according to the manufacturer's instructions (Packard, Meridin, Conn.)

### Results: Mu Opioid Receptor

Mutation of the Asn residue at position 150 of SEQ ID NO: 1 to Ala yielded a constitutively active rat mu opioid receptor. In FIG. 6 and Table 1, below, the results of the wild-type and Asn150Ala mutant rat mu opioid receptors are compared side by side. The basal activity of the wild-type rat mu opioid receptor approximates the basal activity of the negative control vector (pcDNA 1 lacking any encoded gene). In contrast, there is a significant increase (approximately 6.5 fold) in basal activity of the Asn150Ala mutant mu opioid receptor, indicating that the mutant mu opioid receptor is constitutively active.

TABLE 1

| | Average Basal | |
|---|---|---|
| Receptor | Activity | Average Ligand Stimulated |
| Activity | (Light Emission) | (Light Emission) |
| pcDNA 1 | 16,041 | 16,746 |
| (SRE + Gq5i) | | |
| wild-type rat mu opioid | 8,436 | 87,461 |
| receptor | | |
| (SRE + Gq5i) | | |
| Asn150Ala rat mu opioid | *56,498 | 86,996 |
| receptor | | |
| (SRE + Gq5i) | | |

*6.5-fold stimulation of basal level activity.

### Example 2

### Cholecystokinin-B/Ga strin Receptor (CCK-BR)

This example describes the identification of a constitutively active CCK-BR receptor, as adopted from Beinborn et al. (J. Biol. Chem. 273(23): 14146-14151 (1998) and Beinborn et al., Gastroenterology 110, (suppl.) A1059) (1996)). In addition, this example demonstrates the success of the inventive assay in detecting the constitutive activity of the mutant CCK-BR.

### Identifying Regions of Homology and Generating Mutant CCK-BR Receptors

Molecular characterization of the third intracellular loop of the human CCK-BR led to the identification of a point mutation (Leu325Glu) that results in constitutive CCK-BR activity (see, Beinborn et al. supra (1996)). Briefly, the strategy was based on the theory that domain swapping between related polypeptides with different second messenger couplings could yield receptors having increased basal activity. Segments of 4-5 amino acids were substituted in the third intracellular loop of the CCK-BR with corresponding sequences from the vasopressin 2 receptor, a protein with 30% amino acid identity to CCK-BR. However, these proteins are coupled to different signal transduction pathways. CCK-BR is coupled to phospholipase C activation, whereas the vasopressin 2 receptor is coupled to adenylyl cyclase as the predominant signal transduction pathway (Beinborn et al., supra (1996)).

### Assaying Mutant CCK-BR Receptors for Constitutive Activity

As described in Beinborn et al., recombinant receptors were transiently expressed in COS-7 cells and ligand affinities were assessed by ¹²⁵I CCK-8 competition binding experiments. In addition, phospholipase C-mediated production of inositol phosphate was measured in the absence and in the presence of agonists. One of the block substitutions from the vasopressin 2 receptor, 250AHVSA, conferred agonist-independent constitutive activity when introduced into the corresponding region of the third intracellular loop of the CCK-BR. The mutant CCK-BR triggered a 10-fold higher basal turnover of inositol phosphate compared to wild-type CCK-BR. Substitution of 253SA and even 253S alone within the same segment was sufficient to confer constitutive activity as well (Beinborn et al., (Abstract) supra (1996).)

Additional studies were carried out as described in Beinborn et al. (supra (1998)). In particular, the Leu325Glu CCK-BR mutant triggers constitutive production of inositol phosphates to levels exceeding wild-type CCK-BR (Beinborn et al., FIG. 1A supra (1998)). Briefly, the human wild-type CCK-BR and the constitutively active Leu325Glu CCK-BR mutant were transiently expressed in COS-7 cells. Control cells (“no receptor”) were transfected with the empty expression vector, pcDNA1. Cells were pre-labeled overnight with myo-[³H]inositol and then stimulated with ligand for 30 minutes in the presence of 10 mM LiCl. The constitutively active CCK-BR mutant is clearly distinguished from the wild-type receptor by its ability to trigger inositol phosphate production in the absence of agonist.

In order to demonstrate that the assay of the present invention could be used to detect constitutive activity of the Leu325Glu CCK-Br mutant successfully, we performed luciferase assays to measure the constitutive activity of the Leu325Glu CCK-BR mutant. HEK293 cells were transfected (as described above) with SMS-Luc and an expression vector encoding any one of pcDNA1, wild-type CCK-BR, or Leu325Glu CCK-BR. As demonstrated in the left panel of FIG. 2, the Leu325Glu CCK-BR mutant has increased basal level activity compared to the wild-type CCK-BR.

### Example 3

### Constitutively Active Melanocortin-4 Receptor

This example describes the identification of a constitutively active melanocortin-4 (MC-4) receptor.

### Identifying Regions of Homology and Generating MC-4 Receptor Mutants

As shown in FIG. 9, the “DRY” motif is conserved between the Class I G protein-coupled oxytocin, vasopressin-V-2, cholecystokinin-A (CCK-A), melanocortin-4 (MC-4), and α_{1B} adrenergic receptors (FIG. 9). Based on this homology, plus precedent that substitution of aspartic acid within the DRY motif results in constitutively active oxytocin, vasopressin V-2, CCK-A, and α1B receptors, we hypothesized that substitution of the D (Asp) residue at position 146 of MC-4 by a non-charged residue would yield a constitutively active receptor (the MC-4 sequence is available as Genebank Accession is L08603). An Asp146Met mutant MC-4 receptor was generated using routine methods.

### Assay of Mutant MC-4 Receptors for Constitutive Activity

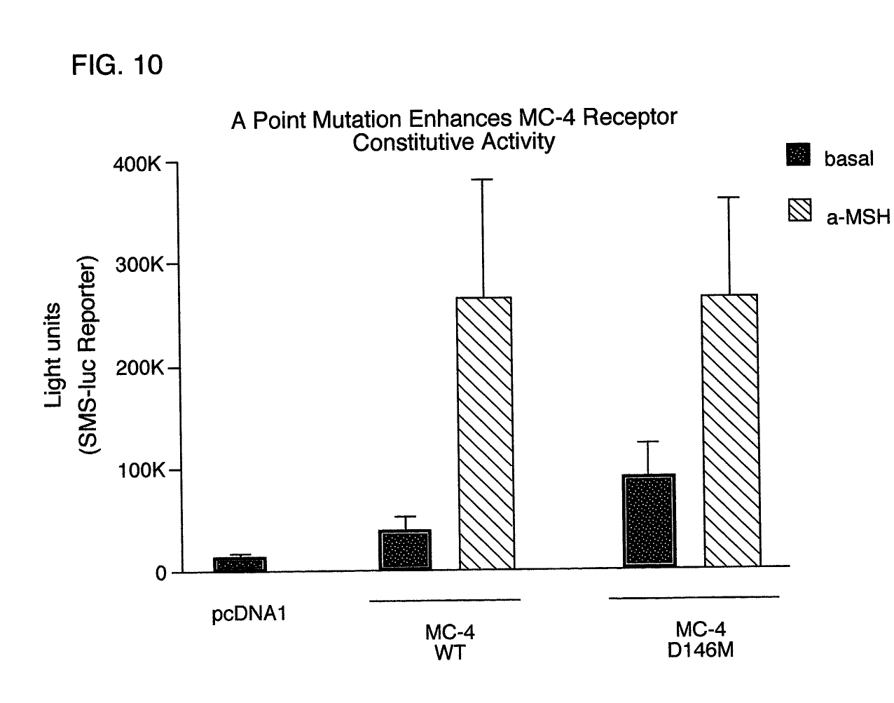

As demonstrated in FIG. 10, the assay of the present invention was capable of detecting constitutive activity of the mutant Asp146Met MC-4 receptor. Briefly, HEK293 cells were cotransfected, as described above, with an expression vector encoding either the wild-type MC-4 receptor or the Asp146Met mutant MC-4 receptor and the reporter construct, SMS-Luc. As a negative control, cells were transfected with SMS-Luc and pcDNA1. Basal and ligand (αMHS) induced activity of the negative control, the wild-type MC-4 receptor, and the Asp146Met mutant MC-4 receptor were measured using the luciferase assay described above. The Asp146Met mutant MC-4 receptor mutant clearly exhibited a higher basal level activity than its wild-type counterpart.

### Other Embodiments

One of ordinary skill in the art would also appreciate that the assay of the present invention is not limited to the identification of constitutively active G protein-coupled receptors, but may be extended to the identification other types of receptors, for example, single transmembrane receptors and nuclear receptors.

All references cited herein are hereby incorporated by reference.

### Appendix 1

### G Protein Chimera Users Manual

### Introduction

Since the first description of G protein chimeras that can alter the signaling phenotype of receptors, many investigators have found them useful for a variety of research purposes. Several people who work with Gᵢ-coupled receptors have found that it is easier to study the stimulation of phospholipase C than the inhibition of adenylyl cyclase. Several groups have used the chimeras to develop rapid assays of receptor activation that can be used for screening mutants or agonist drugs. Others have used the chimeras to complement mutant receptors in detailed structure-function studies.

Over the past four years, I have sent over sixty samples of chimeras. The collection of chimeras has gradually grown and has been improved by the addition of epitope-tagged versions. This update should help people use the chimeras most effectively. Many people who received the original clones may want to upgrade to the new versions.

### Structure-Function Studies with G Proteins

The carboxyl-terminus of the G alpha protein subunit is a key determinant of receptor specificity. We have previously shown that the Gq alpha subunit (alpha q) can be made to respond to Gi alpha-coupled receptors by replacing its carboxyl-terminus with the corresponding Gi2 alpha, Go alpha, or Gz alpha residues (2). We have recently extended these findings in three ways: 1.C-terminal mutations of Gq alpha/Gi alpha chimeras show that the critical amino acids are in the -3 and -4 positions. 2.Exchange of carboxyl-termini between Gq alpha and Gs alpha allows activation by receptors appropriate to the C-terminal residues. 3.We identify receptors that either do or do not activate the expected C-terminal chimeras (Gq alpha/Gi alpha, Gq alpha/Os alpha, Gs alpha/Gq alpha). Replacement of the five carboxyl-terminal amino acids of Gq alpha with the Gs alpha sequence permitted an Gs alpha-coupled receptor (the V2 vasopressin receptor, but not the beta 2-adrenoceptor) to stimulate phospholipase C. Replacement of the five carboxyl-terminal amino acids of Gs alpha with residues of Gq alpha permitted certain Gq alpha-coupled receptors (bombesin and V1a vasopressin receptors, but not the Oxytocin receptor) to stimulate adenylyl cyclase. Thus, the relative importance of the G alpha carboxyl-terminus for permitting coupling to a new receptor depends on the receptor with which it is paired. These studies refine our understanding of the basis of receptor-G alpha specificity. Substitutions of the C-termini of Gq alpha and other G-alpha subunits has recently been instrumental in developing high throughput screens for new agonists of G protein-coupled receptors [Broach J. R. and Thorner J. (1996) High-throughput screening for drug discovery. Nature 384 (Suppl.):14-16].

### Chimera Summary

Notes on the chimeras:

1. All have been subcloned into pcDNA-1, in the Bam HI/Nsi I cassette with q4WT as parent construct for the “q” chimeras and Gs-WT-HA as the parent construct for the “s” chimeras (see below for the description of the parent constructs).

2. All have the internal HA epitope, which does not affect receptor coupling, yet allows recognition by the 12CA5 antibody (available from Boehringer Mannheim as a purified monoclonal and directly conjugated to HRP, which is convenient for Westerns).

3. All the constructs are in pcDNA-1 which require sup F selection for Amp and Tet resistance. This requires special competent bacteria that are available in most labs, but can also be purchased from Invitrogen (for example, mc1061/p3).

qi5-HA: This is Gq alpha with the C-terminal amino acids changed from Gq alpha to Gi alpha residues (EYNLV to DCGLF). This construct allows many Gi-coupled receptors to stimulate phospholipase C (PLC). This is the most popular chimera, perhaps because it is easier to talk about coupling to Gi-coupled receptors with “qi5” rather than “qo5” or “q25.”

Click here to see sequence for qi5.

qo5-HA: This is Gq alpha with the C-terminal amino acids changed from Gq alpha to Go alpha residues (EYNLV to GCGLY). Works the same as qi5 but (for unknown reasons) has a slightly lower basal PLC activity. This can increase the signal-to-noise ratio, so I tend to use it the most.

Click here to see sequence for qo5.

qz5-HA: This is Gq alpha with the C-terminal amino acids changed from Gq alpha to Gz alpha residues (EYNLV to YIGLC). Works the same as qi5 and is the least popular since no one knows what Gz alpha really does in nature. Since qz5 is not sensitive to pertussis toxin, qz5 may be the only G protein activated by a Gi-coupled receptor in cells treated with pertussis toxin. This trick can be experimentally useful in settings where you want experimental control of the exact G protein and the receptor that is activated. It is theoretically possible that this construct will work better than the other constructs for particular receptors, but I have not seen this happen yet.

qs5-HA: This is Gq alpha with the C-terminal amino acids changed from Gq alpha to Gs alpha residues (EYNLV to QYELL). This construct allows some Gs-coupled receptors to stimulate phospholipase C.

Click here to see sequence for qs5.

sq5-HA: This is Gs alpha with the C-terminal amino acids changed from Gs alpha to Gq alpha residues (QYELL to EYNLV). This construct allows some Gq-coupled receptors to stimulate Adenylate cyclase. There isn't much experience with this chimera at the moment. It may be useful for people who find tie AC stimulation is better readout of receptor activation than PLC stimulation.

13Z: This is G13 alpha with the C-terminal amino acids changed from G13 alpha to Gz alpha residues (QLMLQ to YIGLC). This construct allows some Gi-coupled receptors to stimulate an increase in pH of cells. There isn't much experience with this chimera at the moment, but it was used successfully with the D2-dopamine receptor [see Voyno-Yasenetskaya et al. (1994) JBC 269:4721-4724].

Please note that this construct is not epitope tagged since no one has made a reliable internal tag for G13 alpha.

### Parent Constructs

q4WT: This is Gq alpha with an HA epitope engineered into an internal site that does not seem to affect receptor coupling in multiple studies. Epitope tagged by Paul Wilson, see Wedegaertner, JBC268: 25001-25008. The 5″ non-coding sequences were removed, but the 3′ non-coding sequences remain, as in Strathmann & Simon (1990) PNAS 87:9113-9117. The parent construct has been donated to the ATCC by the Bourne Lab.

Click here to see sequence for q4WT

Gs-WT-HA: This construct is also known as “GSL” in the Bourne Lab. This is “wild type” Gs alpha in pcDNA-1 with a HA epitope engineered into an internal site that does not seem to affect receptor coupling in multiple studies. [See Levis & Bourne (1992) J. Cell Biol. 119:1297-1307] The parent construct has been donated to the ATCC by the Bourne Lab.

Click here to see sequence for Gs-WT-RA.

Below is a list of publications that describes how we have used the G alpha C-terminal chimeras

Initial Description of Chimeras

1. Conklin B. R., Farfel Z., Lustig K. D., Julius D. and Bourne H. R. (1993) Substitution of three amino acids switches receptor specificity of Gq alpha to that of Gi alpha. Nature 363:274-276

2. Conklin B. R., Herzmark P., Ish ida S., Voyno-Yasenetskaya T. A., Sun Y. and Bourne H. R. (1996) C-Terminal mutations of Gq alpha and Gs alpha that alter the fidelity of receptor activation. Mol Pharmacol. 50:885-890.

3. Voyno-Yasenetskaya T., Conklin B. R., Gilbert R. L., Hooley R., Bourne H. R. and Barber D. L. (1994) G13 alpha stimulates Na-H Exchange. J. Biol. Chem. 269:4721-4724.

Chimeras Used in Recent Studies

1. Liu J., Conklin B. R., Blin N., Yun J., Wess J. (1995) Identification of a receptor/G-protein contact site critical for signaling specificity and G-protein activation. Proceedings of the National Academy of Sciences, U.S.A. 92:11642-11646.

2. Messier T. L., Dorman C. M., Bräuner-Osborne H., Eubanks D. and Brann M. R. (1995) High throughput assays of cloned adrenergic, muscarinic, neurokinin, and neurotrophin receptors in living mammalian cells. Pharmacol. Toxicol. 76:308-311.

3. Liu J., Blin N., Conklin B. R. and Wess J. (1996) Molecular mechanisms involved in muscarinic acetylcholine receptor-mediated G protein activation studied by insertion mutagenesis. J. Biol. Chem. 271:6172-6178.

4. Boss V., Talpade D. J. and Murphy T. J (1996) Induction of NFAT-mediated transcription by Gq-coupled receptors in lymphoid and non-lymphoid cells. J. Biol Chem. 271:10429-10432.

5. Arai H. and Charo I. F. (1996) Differential regulation of G-protein-mediated signaling by chemokine receptors. J. Biol. Chem 271:21814-21819

6. Liu J., Blin N., Conklin B. R. and Wess J. (1996) Molecular mechanisms involved in muscarinic acetylcholine receptor-mediated G protein activation studied by insertion mutagenesis, J. Biol. Chem, 271;6172-6178.

7. Gomeza J., Mary S., Brabet I., Parmentier M. -L, Restituito S., Bockaert J. and Pin J. -P. (1996) Coupling of metabotropic glutamate receptors 2 and 4 to G 15 alpha, G16 alpha, and chimeric Gqalpha/Gi alpha proteins: Characterization of new antagonists. Mol. Pharmacol. 50:923-930.

8. Parmentier M. -L., Pin J. -P., Bockaert J. and Grau Y. (1996) Cloning and functional expression of a drosophila metabotropic glutamate receptor expressed in the embryonic central nervous system. J. Neurosci. 16:6687-6694.

9. Burstein E. S., Bräuner-Osborne H., Spalding T. A., Conklin B. R. and Brann M. R. (1997) Interactions of muscarinic receptors with the heterotrimeric G proteins Gq and G12: Transduction of proliferative signals. J. Neurochem. 68:525-533.

10. Komatsuzaki K., Murayama Y., Gimabarella U., Ogata E., Seino S. and Nishimoto L (1997) A novel system that reports the G-proteins linked to a given receptor: A study of type 3 somatostatin receptor. FEBS Lett. 406:165-170.

11. Kostenis E., Gomeza J., Lerche C. and Wess J. (1997) Genetic analysis of receptor-Gaq coupling selectivity. J. Biol. Chem. 272:23675-37681,

12. Monteclaro F. S., Arai H. and Charo I. F. (1997) Molecular approached to identifying ligand binding and signaling domains of c-c chemokine receptors. Methods Enzymol. 288:70-84.

13. Tsu R. C., Ho M. K. C., Yung L. Y., Joshi S. and Wong Y. H. (1997) Role of amino- and carboxyl-terminal regions of Gaz in the recognition of Gi-coupled receptors. Mol. Pharmacol. 52:38-45.

14. Coward P., Wada H. G., Falk M. S., Chan S. D. H., Meng F., Akil H. and Conklin B. R. (1998) Controlling signaling with a specifically designed Gi-coupled receptor. Proc. Natl. Acad. Sci. 95:352-357.

15. Ancellin, N., and Hla, T. (1999) Differential pharmacological properties and signal transduction of the sphingosine 1-phosphate receptors EDG-1, EDG-3, and EDG-5. J. Biol. Chem. 274: 18997-19002.

I hope this information is useful. Please send preprints of papers that use the chimeras, and feel free to contact me if you have suggestions.

Mailing Address:

Bruce R. Conklin, M. D.

The Gladstone Institutes of Neurological and Cardiovascular Disease

Departments of Medicine and Pharmacology, UCSF

P.O. Box 419100

San Francisco, Calif. 94141-9100

Other information:

Office: (415) 695-3758

Lab: (415) 695-3784

Fax: (415) 285-5632

bconklin @gladstone.ucsf.edu

For further information, see Conklin Lab Technical Information

Return to Overview of Research Interests

Return to Conklin H Home Page

Last modified: October 1998

1. A method of identifying a polymorphic receptor having altered signaling, comprising the steps of:
a) cotransfecting a first host cell with a reporter construct and an expression vector, said reporter construct comprising a response element and a promoter operably linked to a reporter gene, said response element being sensitive to a signal induced by said receptor, and said expression vector comprising a promoter operably linked to a candidate receptor having a genetic polymorphism;
b) cotransfecting a second host cell with said reporter construct and a negative control vector; and
c) measuring the level of expression of said reporter construct in said first host cell and said second host cell, an increased or decreased level of expression in the first host cell compared to the second host cell identifying said candidate receptor as a polymorphic receptor having altered signaling.

2. The method of claim 1, wherein said signaling is ligand dependent signaling.

3. The method of claim 1, wherein said signaling is ligand independent signaling.

4. The method of claim 1, wherein said polymorphic receptor having altered signaling is a polymorphic receptor having an increase or decrease in basal signaling.

5. The method of claim 1, wherein said polymorphic receptor having altered signaling is a polymorphic receptor having an increased or decreased sensitivity to ligand induced signaling.

6. The method of claim 1, wherein said polymorphic receptor having altered signaling is a polymorphic receptor having increased or decreased potency.

7. The method of claim 1, wherein said polymorphic receptor having altered signaling is a polymorphic receptor having an absence of signaling.

8. The method of claim 1, wherein said polymorphic receptor having altered signaling is a G protein-coupled receptor.

9. The method of claim 8, wherein said G protein-coupled receptor is coupled to a G protein selected from the group consisting of Gαq, Gαs, and Gαi.

10. The method of claim 1, wherein said polymorphic receptor having altered signaling is a single transmembrane receptor.

11. The method of claim 10, wherein said single transmembrane receptor is an erythropoietin receptor.

12. The method of claim 1, wherein said polymorphic receptor having altered signaling is a nuclear receptor.

13. The method of claim 12, wherein said nuclear receptor is a steroid hormone receptor.

14. The method of claim 1, wherein said polymorphic receptor having altered signaling is further screened for an alteration in ligand induced response.

15. The method of claim 14, wherein said ligand is a drug.

16. The method of claim 1, wherein, in step (c), the basal level of expression of said reporter construct is measured in said first host cell and said second host cell, and an increased basal level of expression in said first host cell compared to said second host cell identifies said polymorphic receptor as a constitutively active receptor.

17. The method of claim 1, wherein said measuring is accomplished using a transcriptional reporter assay.

18. The method of claim 1, wherein said response element is selected from the group consisting of the somatostatin promoter element, the serum response element, and the cAMP response element.

19. The method of claim 1, wherein said receptor is naturally occurring.

20. The method of claim 1, wherein said polymorphic receptor is a constitutively active receptor.

21. The method of claim 1, wherein said polymorphic receptor is a hypersensitive or hyposensitive receptor.

22. The method of claim 1, wherein said polymorphic receptor is a non-functional receptor.

23. A method of identifying a G protein-coupled receptor with altered signaling, said method comprising:
a) co-transfecting a first host cell with:
i) a reporter construct, said reporter construct comprising a G protein response element and a promoter operably linked to a reporter gene,
ii) a first expression vector, said first expression vector comprising a promoter operably linked to a candidate G protein-coupled receptor, and
iii) a second expression vector, said second expression vector comprising a promoter operably linked to a chimeric G protein, wherein said chimeric G protein is capable of receiving a signal from said candidate G protein-coupled receptor and increasing the expression of said reporter construct;
b) co-transfecting a second host cell with said reporter construct, said second expression vector, and a negative control vector; and
c) measuring the level of expression of said reporter construct in said first host cell and said second host cell,
wherein an increased or decreased level of expression in the first host cell compared to the second host cell identifies said candidate receptor as a G protein-coupled receptor with altered signaling.

24. The method of claim 23, wherein said chimeric G protein comprises a G protein with the C-terminal 3 amino acids changed to those of another G protein.

25. The method of claim 23, wherein chimeric G protein is selected from the group consisting of Gq5i, Gq5o, Gq5z, Gq5s, Gs5q, and G13Z.

26. The method of claim 23, wherein said reporter construct is selected from the group consisting of a luciferase construct, a beta-galactosidase construct, and a chloramphenicol acetyl transferase construct.

27. The method of claim 23, wherein reporter construct is a luciferase construct.

28. The method of claim 23, wherein said response element is selected from the group consisting of the somatostatin promoter, the serum response element, and the cAMP response element.

29. The method of claim 23, wherein said G protein coupled receptor is selected from the group consisting of a constitutively active receptor, a hypersensitive receptor, a hyposensitive receptor, a non-functional receptor, a silent receptor, and a partially silent receptor.

30. The method of claim 23, wherein said G protein-coupled receptor is coupled to a G protein selected from the group consisting of Gαq, Gαs, GαI, and Go.

31. The method of claim 23, wherein said signaling is ligand dependent signaling.

32. The method of claim 23, wherein said signaling is ligand independent signaling.

33. The method of claim 23, wherein said G protein coupled receptor is further screened for an alteration in a response induced by a ligand.

34. The method of claim 33, wherein said ligand is selected from the group consisting of a drug, an agonist, an antagonist, and an inverse agonist.

35. A method of identifying a receptor having decreased signaling activity, comprising the steps of:
a) cotransfecting a first host cell with a reporter construct and an expression vector, said reporter construct comprising a response element and a promoter operably linked to a reporter gene, said response element being sensitive to a signal induced by said receptor, and said expression vector comprising a promoter operably linked to a candidate receptor;
b) cotransfecting a second host cell with said reporter construct and a negative control vector; and
c) measuring the level of expression of said reporter construct in said first host cell and said second host cell, a decreased level of expression in the first host cell compared to the second host cell identifying said candidate receptor as a receptor having decreased signaling activity.

36. The method of claim 35, wherein said receptor has no signaling activity.
